SEQUENCE LISTING (58 sequences)

SEQ ID NO: 1 - DNA, 597 bp, Artificial Sequence
  Synthetic
atggtgtggctccccagagttccctgtgtggcagctgtgatcctgttgctgacagtgctgagccctccagtggctttggtcagagactcctacccatacgatgttccagattacgctggaggttccgcggccgcaggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctcctggatccggtggaggcggatcaggtggcggtggaagtggaggtggtggatcttccggacaagtgcagctcctggagtctggggctgagctggtgaggcctgggtcctcagtgaagatttcctgcaaggcttctggctatgcattcagtagctactggatgaactgggtgaagcagaggggtggaggcggttcccatcaccatcaccatcactgatga

SEQ ID NO: 2 - protein, 197 aa, Artificial Sequence
  Synthetic
MVWLPRVPCVAAVILLLTVLSPPVALVRDSYPYDVPDYAGGSAAAGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRPGSSVKISCKASGYAFSSYWMNWVKQRGGGGSHHHHHH

SEQ ID NO: 3 - DNA, 570 bp, Artificial Sequence
  Synthetic
atggtgtggctccccagagttccctgtgtggcagctgtgatcctgttgctgacagtgctgagccctccagtggctttggtcagagactccgagcagaagctgatcagcgaggaggacctgggaggttccgcggccgcaggacagccacccaaactcctcatctatgatgcatccaatctagtttctgggattccacccaggtttagtggcagtgggtctgggacagacttcaccctcaacatccatcctgtggagaaggtggatgctgcaacctatcactgtcagcaaagtactgaagatccgtggacgttcggtggagggaccaagctggaaataaaatctcctggatccggtggaggcggatcaggtggcggtggaagtggaggtggtggatcttccggagagctcgtgctcacccagtctccagcttctttggctgtgtctctagggcagagggccaccatctcctgcaaggccagccaaagtgttgattatgatggtgatagttatttgaactggtaccaacagattggtggaggcggttcccatcaccatcaccatcactgatga

SEQ ID NO: 4 - protein, 188 aa, Artificial Sequence
  Synthetic
MVWLPRVPCVAAVILLLTVLSPPVALVRDSEQKLISEEDLGGSAAAGQPPKLLIYDASNLVSGIPPRFSGSGSGTDFTLNIHPVEKVDAATYHCQQSTEDPWTFGGGTKLEIKSPGSGGGGSGGGGSGGGGSSGELVLTQSPASLAVSLGQRATISCKASQSVDYDGDSYLNWYQQIGGGGSHHHHHH

SEQ ID NO: 5 - DNA, 1203 bp, Artificial Sequence
  Synthetic
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacagcggccgcaggacagccacccaaactcctcatctatgatgcatccaatctagtttctgggattccacccaggtttagtggcagtgggtctgggacagacttcaccctcaacatccatcctgtggagaaggtggatgctgcaacctatcactgtcagcaaagtactgaagatccgtggacgttcggtggagggaccaagctggaaataaaatctcctggatccggtggaggcggatcaggtggcggtggaagtggaggtggtggatcttccggagagctcgtgctcacccagtctccagcttctttggctgtgtctctagggcagagggccaccatctcctgcaaggccagccaaagtgttgattatgatggtgatagttatttgaactggtaccaacagattggtggaggcggttcccatcaccatcaccatcactgatgaagatctcaattggaattctga

SEQ ID NO: 6 - protein, 392 aa, Artificial Sequence
  Synthetic
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTAAAGQPPKLLIYDASNLVSGIPPRFSGSGSGTDFTLNIHPVEKVDAATYHCQQSTEDPWTFGGGTKLEIKSPGSGGGGSGGGGSGGGGSSGELVLTQSPASLAVSLGQRATISCKASQSVDYDGDSYLNWYQQIGGGGSHHHHHH

SEQ ID NO: 7 - DNA, 1293 bp, Artificial Sequence
  Synthetic
atggcttgcaattgtcagttgatgcaggatacaccactcctcaagtttccatgtccaaggatggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacagcggccgcaggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctcctggatccggtggaggcggatcaggtggcggtggaagtggaggtggtggatcttccggacaagtgcagctcctggagtctggggctgagctggtgaggcctgggtcctcagtgaagatttcctgcaaggcttctggctatgcattcagtagctactggatgaactgggtgaagcagaggggtggaggcggttcccatcaccatcaccatcactgatgaagatctcaattggaattctga

SEQ ID NO: 8 - protein, 402 aa, Artificial Sequence
  Synthetic
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTAAAGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRPGSSVKISCKASGYAFSSYWMNWVKQRGGGGSHHHHHH

SEQ ID NO: 9 - DNA, 1227 bp, Artificial Sequence
  Synthetic
atggaccccagatgcaccatgggcttggcaatccttatctttgtgacagtcttgctgatctcagatgctgtttccgtggagacgcaagcttatttcaatgggactgcatatctgccgtgcccatttacaaaggctcaaaacataagcctgagtgagctggtagtattttggcaggaccagcaaaagttggttctgtacgagcactatttgggcacagagaaacttgatagtgtgaatgccaagtacctgggccgcacgagctttgacaggaacaactggactctacgacttcacaatgttcagatcaaggacatgggctcgtatgattgttttatacaaaaaaagccacccacaggatcaattatcctccaacagacattaacagaactgtcagtgatcgccaacttcagtgaacctgaaataaaactggctcagaatgtaacaggaaattctggcataaatttgacctgcacgtctaagcaaggtcacccgaaacctaagaagatgtattttctgataactaattcaactaatgagtatggtgataacatgcagatatcacaagataatgtcacagaactgttcagtatctccaacagcctctctctttcattcccggatggtgtgtggcatatgaccgttgtgtgtgttctggaaacggagtcaatgaagatttcctccaaacctctcaatttcactcaagagtttccatctcctcaaacgtattggaaggaggcggccgcaggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctcctggatccggtggaggcggatcaggtggcggtggaagtggaggtggtggatcttccggacaagtgcagctcctggagtctggggctgagctggtgaggcctgggtcctcagtgaagatttcctgcaaggcttctggctatgcattcagtagctactggatgaactgggtgaagcagaggggtggaggcggttcccatcaccatcaccatcactgatgaagatctcaattggaattctga

SEQ ID NO: 10 - protein, 400 aa, Artificial Sequence
  Synthetic
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEAAAGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRPGSSVKISCKASGYAFSSYWMNWVKQRGGGGSHHHHHH

SEQ ID NO: 11 - DNA, 1953 bp, Artificial Sequence
  Synthetic
atggcttcaacccgtgccaagcccacgctacctctgctcctggccctggtcaccgttgtgatccctgggcctggtgatgctcaggtatccatccatcccagagaagccttcctgccccagggtgggtccgtgcaggtgaactgttcttcctcatgcaaggaggacctcagcctgggcttggagactcagtggctgaaagatgagctcgagagtggacccaactggaagctgtttgagctgagcgagatcggggaggacagcagtccgctgtgctttgagaactgtggcaccgtgcagtcgtccgcttccgctaccatcaccgtgtattcgtttccggagagtgtggagctgagacctctgccagcctggcagcaagtaggcaaggacctcaccctgcgctgccacgtggatggtggagcaccgcggacccagctctcagcagtgctgctccgtggggaggagatactgagccgccagccagtgggtgggcaccccaaggaccccaaggagatcacattcacggtgctggctagcagaggggaccacggagccaatttctcatgccgcacagaactggatctcaggccgcaagggctggcattgttctctaatgtctccgaggccaggagcctccggactttcgatcttccagctaccatcccaaagctcgacacccctgacctcctggaggtgggcacccagcagaagttgttttgctccctggaaggcctgtttcctgcctctgaagctcggatatacctggagctgggaggccagatgccgacccaggagagcacaaacagcagtgactctgtgtcagccactgccttggtagaggtgactgaggagttcgacagaaccctgccgctgcgctgcgttttggagctagcggaccagatcctggagacgcagaggaccttaacagtctacaacttttcagctccggtcctgaccctgagccagctggaggtctcggaagggagccaagtaactgtgaagtgtgaagcccacagtgggtcgaaggtggttcttctgagcggcgtcgagcctaggccacccaccccgcaggtccaattcacactgaatgccagctcggaggatcacaaacgaagcttcttttgctctgccgctctggaggtggcgggaaagttcctgtttaaaaaccagaccctggaactgcacgtgctgtatggtcctcggctggacgagacggactgcttggggaactggacctggcaagaggggtctcagcagactctgaaatgccaggcctgggggaacccatctcctaagatgacctgcagacggaaggcagatggtgccctgctgcccatcggggtggtgaagtctgtcaaacaggagatgaatggtacatacgtgtgccatgcctttagctcccatgggaatgtcaccaggaatgtgtacctgacagtactgtaccactctcaaaataactggactgcggccgcaggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctcctggatccggtggaggcggatcaggtggcggtggaagtggaggtggtggatcttccggacaagtgcagctcctggagtctggggctgagctggtgaggcctgggtcctcagtgaagatttcctgcaaggcttctggctatgcattcagtagctactggatgaactgggtgaagcagaggggtggaggcggttcccatcaccatcaccatcactgatgaagatctcaattggaattctga

SEQ ID NO: 12 - protein, 642 aa, Artificial Sequence
  Synthetic
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTAAAGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRPGSSVKISCKASGYAFSSYWMNWVKQRGGGGSHHHHHH

SEQ ID NO: 13 - DNA, 1254 bp, Artificial Sequence
  Synthetic
atggctacaaggctcctctgttacacagtactttgtctcctgggtgcaagaattttgaattgtatcaaagaggaacacaccatcatccaggcagagttctatcttttaccagacaaacgtggagagtttatgtttgactttgacggcgatgagattttccatgtagacattgaaaagtcagagaccatctggagacttgaagaatttgcaaagtttgccagctttgaggctcagggtgcactggctaatatagctgtggacaaagctaacctggatgtcatgaaagagcgttccaacaacactccagatgccaacgtggccccagaggtgactgtactctccagaagccctgtgaacctgggagagcccaacatcctcatctgtttcattgacaagttctcccctccagtggtcaatgtcacctggctccggaatggacggcctgtcaccgaaggcgtgtcagagacagtgtttctcccgagggacgatcacctcttccgcaaattccactatctgaccttcctgccctccacagatgatttctatgactgtgaggtggatcactggggcttggaggagcctctgcggaagcactgggagtttgaagagaaaaccctcctcccagaaactaaagagtctagaggtggcctggaagttctgttccaggggcccgaattcggcggttccactacagctccatcagctcagctcgaaaaagagctccaggccctggagaaggaaaatgcacagctggaatgggagttgcaagcactggaaaaggaactggctcaggcggccgcaggacagccacccaaactcctcatctatgatgcatccaatctagtttctgggattccacccaggtttagtggcagtgggtctgggacagacttcaccctcaacatccatcctgtggagaaggtggatgctgcaacctatcactgtcagcaaagtactgaagatccgtggacgttcggtggagggaccaagctggaaataaaatctcctggatccggtggaggcggatcaggtggcggtggaagtggaggtggtggatcttccggagagctcgtgctcacccagtctccagcttctttggctgtgtctctagggcagagggccaccatctcctgcaaggccagccaaagtgttgattatgatggtgatagttatttgaactggtaccaacagattggtggaggcggttcccatcaccatcaccatcactgatgaagatctcaattggaattctga

SEQ ID NO: 14 - protein, 409 aa, Artificial Sequence
  Synthetic
MATRLLCYTVLCLLGARILNCIKEEHTIIQAEFYLLPDKRGEFMFDFDGDEIFHVDIEKSETIWRLEEFAKFASFEAQGALANIAVDKANLDVMKERSNNTPDANVAPEVTVLSRSPVNLGEPNILICFIDKFSPPVVNVTWLRNGRPVTEGVSETVFLPRDDHLFRKFHYLTFLPSTDDFYDCEVDHWGLEEPLRKHWEFEEKTLLPETKESRGGLEVLFQGPEFGGSTTAPSAQLEKELQALEKENAQLEWELQALEKELAQAAAGQPPKLLIYDASNLVSGIPPRFSGSGSGTDFTLNIHPVEKVDAATYHCQQSTEDPWTFGGGTKLEIKSPGSGGGGSGGGGSGGGGSSGELVLTQSPASLAVSLGQRATISCKASQSVDYDGDSYLNWYQQIGGGGSHHHHHH

SEQ ID NO: 15 - DNA, 1428 bp, Artificial Sequence
  Synthetic
atggctacaaggctcctctgttacacagtactttgtctcctgggtgcaagaattttgaattgtgcggatccctccggctccgccaacgagagggccgacctgatcgcctacctgaagcaggccaccaaggaattcagatccggaggcggaggctccctggtgcctcggggctccggaggcggaggctccgtcgacagaccatggtttttggaatactgtaaatctgagtgtcatttctacaacgggacgcagcgcgtgcggcttctggtaagatacttctacaacctggaggagaacctgcgcttcgacagcgacgtgggcgagttccgcgcggtgaccgagctggggcggccagacgccgagaactggaacagccagccggagttcctggagcaaaagcgggccgaggtggacacggtgtgcagacacaactatgagatcttcgataacttccttgtgccgcggagagttgagcctacggtgactgtgtaccccacaaagacgcagcccctggaacaccacaacctcctggtctgctctgtgagtgacttctaccctggcaacattgaagtcagatggttccggaatggcaaggaggagaaaacaggaattgtgtccacgggcctggtccgaaatggagactggaccttccagacactggtgatgctggagacggttcctcagagtggagaggtttacacctgccaggtggagcatcccagcctgaccgaccctgtcacggtcgagtggaaagcacagtccacatctgcacagaacaagtctagaggtggcctggaagttctgttccaggggcccgaattcggcggttccactacagctccatcagctcagttgaaaaagaaattgcaagcactgaagaaaaagaacgctcagctgaagtggaaacttcaagccctcaagaagaaactcgcccaggcggccgcaggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctcctggatccggtggaggcggatcaggtggcggtggaagtggaggtggtggatcttccggacaagtgcagctcctggagtctggggctgagctggtgaggcctgggtcctcagtgaagatttcctgcaaggcttctggctatgcattcagtagctactggatgaactgggtgaagcagaggggtggaggcggttcccatcaccatcaccatcactgatgaagatctcaattggaattctga

SEQ ID NO: 16 - protein, 467 aa, Artificial Sequence
  Synthetic
MATRLLCYTVLCLLGARILNCADPSGSANERADLIAYLKQATKEFRSGGGGSLVPRGSGGGGSVDRPWFLEYCKSECHFYNGTQRVRLLVRYFYNLEENLRFDSDVGEFRAVTELGRPDAENWNSQPEFLEQKRAEVDTVCRHNYEIFDNFLVPRRVEPTVTVYPTKTQPLEHHNLLVCSVSDFYPGNIEVRWFRNGKEEKTGIVSTGLVRNGDWTFQTLVMLETVPQSGEVYTCQVEHPSLTDPVTVEWKAQSTSAQNKSRGGLEVLFQGPEFGGSTTAPSAQLKKKLQALKKKNAQLKWKLQALKKKLAQAAAGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRPGSSVKISCKASGYAFSSYWMNWVKQRGGGGSHHHHHH

SEQ ID NO: 17 - DNA, 618 bp, Artificial Sequence
  Synthetic
atggtgtggctccccagagttccctgtgtggcagctgtgatcctgttgctgacagtgctgagccctccagtggctttggtcagagactcctacccatacgatgttccagattacgctggaggttccgcggccgcagggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctcctggatccggtggaggcggatcaggtggcggtggaagtggaggtggtggatcttccggacaagtgcagctcctggagtctggggctgagctggtgaggggtggaggcggttcccatcaccatcaccatcactgatgaagatctcaattggaattctga

SEQ ID NO: 18 - protein, 197 aa, Artificial Sequence
  Synthetic
MVWLPRVPCVAAVILLLTVLSPPVALVRDSYPYDVPDYAGGSAAAGSSVKISCKASGYAFSSYWMNWVKQRPGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRGGGGSHHHHHH

SEQ ID NO: 19 - DNA, 594 bp, Artificial Sequence
  Synthetic
atggtgtggctccccagagttccctgtgtggcagctgtgatcctgttgctgacagtgctgagccctccagtggctttggtcagagactccgagcagaagctgatcagcgaggaggacctgggaggttccgcggccgcagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctggaaataaaatctcctggatccggtggaggcggatcaggtggcggtggaagtggaggtggtggatcttccggagagctcgtgctcacccagtctccagcttctttggctgtgtctctaggtggaggcggttcccatcaccatcaccatcactgatgaagatctcaattggaattctga

SEQ ID NO: 20 - protein, 189 aa, Artificial Sequence
  Synthetic
MVWLPRVPCVAAVILLLTVLSPPVALVRDSEQKLISEEDLGGSAAAGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKSPGSGGGGSGGGGSGGGGSSGELVLTQSPASLAVSLGGGGSHHHHHH

SEQ ID NO: 21 - DNA, 1233 bp, Artificial Sequence
  Synthetic
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacagcggccgcagggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctcctggatccggtggaggcggatcaggtggcggtggaagtggaggtggtggatcttccggacaagtgcagctcctggagtctggggctgagctggtgaggggtggaggcggttcccatcaccatcaccatcactgatgaagatctcaattggaattctga

SEQ ID NO: 22 - protein, 402 aa, Artificial Sequence
  Synthetic
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTAAAGSSVKISCKASGYAFSSYWMNWVKQRPGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRGGGGSHHHHHH

SEQ ID NO: 23 - DNA, 1206 bp, Artificial Sequence
  Synthetic
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacagcggccgcagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctggaaataaaatctcctggatccggtggaggcggatcaggtggcggtggaagtggaggtggtggatcttccggagagctcgtgctcacccagtctccagcttctttggctgtgtctctaggtggaggcggttcccatcaccatcaccatcactgatgaagatctcaattggaattctga

SEQ ID NO: 24 - protein, 393 aa, Artificial Sequence
  Synthetic
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTAAAGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKSPGSGGGGSGGGGSGGGGSSGELVLTQSPASLAVSLGGGGSHHHHHH

SEQ ID NO: 25 - DNA, 1227 bp, Artificial Sequence
  Synthetic
atggaccccagatgcaccatgggcttggcaatccttatctttgtgacagtcttgctgatctcagatgctgtttccgtggagacgcaagcttatttcaatgggactgcatatctgccgtgcccatttacaaaggctcaaaacataagcctgagtgagctggtagtattttggcaggaccagcaaaagttggttctgtacgagcactatttgggcacagagaaacttgatagtgtgaatgccaagtacctgggccgcacgagctttgacaggaacaactggactctacgacttcacaatgttcagatcaaggacatgggctcgtatgattgttttatacaaaaaaagccacccacaggatcaattatcctccaacagacattaacagaactgtcagtgatcgccaacttcagtgaacctgaaataaaactggctcagaatgtaacaggaaattctggcataaatttgacctgcacgtctaagcaaggtcacccgaaacctaagaagatgtattttctgataactaattcaactaatgagtatggtgataacatgcagatatcacaagataatgtcacagaactgttcagtatctccaacagcctctctctttcattcccggatggtgtgtggcatatgaccgttgtgtgtgttctggaaacggagtcaatgaagatttcctccaaacctctcaatttcactcaagagtttccatctcctcaaacgtattggaaggaggcggccgcagggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctcctggatccggtggaggcggatcaggtggcggtggaagtggaggtggtggatcttccggacaagtgcagctcctggagtctggggctgagctggtgaggggtggaggcggttcccatcaccatcaccatcactgatgaagatctcaattggaattctga

SEQ ID NO: 26 - protein, 400 aa, Artificial Sequence
  Synthetic
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEAAAGSSVKISCKASGYAFSSYWMNWVKQRPGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRGGGGSHHHHHH

SEQ ID NO: 27 - DNA, 1200 bp, Artificial Sequence
  Synthetic
atggaccccagatgcaccatgggcttggcaatccttatctttgtgacagtcttgctgatctcagatgctgtttccgtggagacgcaagcttatttcaatgggactgcatatctgccgtgcccatttacaaaggctcaaaacataagcctgagtgagctggtagtattttggcaggaccagcaaaagttggttctgtacgagcactatttgggcacagagaaacttgatagtgtgaatgccaagtacctgggccgcacgagctttgacaggaacaactggactctacgacttcacaatgttcagatcaaggacatgggctcgtatgattgttttatacaaaaaaagccacccacaggatcaattatcctccaacagacattaacagaactgtcagtgatcgccaacttcagtgaacctgaaataaaactggctcagaatgtaacaggaaattctggcataaatttgacctgcacgtctaagcaaggtcacccgaaacctaagaagatgtattttctgataactaattcaactaatgagtatggtgataacatgcagatatcacaagataatgtcacagaactgttcagtatctccaacagcctctctctttcattcccggatggtgtgtggcatatgaccgttgtgtgtgttctggaaacggagtcaatgaagatttcctccaaacctctcaatttcactcaagagtttccatctcctcaaacgtattggaaggaggcggccgcagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctggaaataaaatctcctggatccggtggaggcggatcaggtggcggtggaagtggaggtggtggatcttccggagagctcgtgctcacccagtctccagcttctttggctgtgtctctaggtggaggcggttcccatcaccatcaccatcactgatgaagatctcaattggaattctga

SEQ ID NO: 28 - protein, 391 aa, Artificial Sequence
  Synthetic
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEAAAGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKSPGSGGGGSGGGGSGGGGSSGELVLTQSPASLAVSLGGGGSHHHHHH

SEQ ID NO: 29 - DNA, 963 bp, Artificial Sequence
  Synthetic
tccggagggcccacaatcaagccctgtcctccatgcaaatgcccagcacctaacctcttgggtggaggatccggccagccacccaaactcctcatctatgatgcatccaatctagtttctgggattccacccaggtttagtggcagtgggtctgggacagacttcaccctcaacatccatcctgtggagaaggtggatgctgcaacctatcactgtcagcaaagtactgaagatccgtggacgttcggtggagggaccaagctggaaataaaatctcccggttcaggaggaggtggctccggtggaggagggtctggcggaggaggctcaagcggagagctcgtgctcacccagtctccagcttctttggctgtgtctctagggcagagggccaccatctcctgcaaggccagccaaagtgttgattatgatggtgatagttatttgaactggtaccaacagattccaggaagcggctctggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctccagggagtggcggcggcggatccggaggaggcgggtcaggtggcggcggctcatctgggcaagtgcagctcctggagtctggggctgagctggtgaggcctgggtcctcagtgaagatttcctgcaaggcttctggctatgcattcagtagctactggatgaactgggtgaagcagaggggtggaggcggttcccatcaccatcaccatcactgatgaagatctcaattggaattctga

SEQ ID NO: 30 - protein, 312 aa, Artificial Sequence
  Synthetic
SGGPTIKPCPPCKCPAPNLLGGGSGQPPKLLIYDASNLVSGIPPRFSGSGSGTDFTLNIHPVEKVDAATYHCQQSTEDPWTFGGGTKLEIKSPGSGGGGSGGGGSGGGGSSGELVLTQSPASLAVSLGQRATISCKASQSVDYDGDSYLNWYQQIPGSGSGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRPGSSVKISCKASGYAFSSYWMNWVKQRGGGGSHHHHHH

SEQ ID NO: 31 - DNA, 1713 bp, Artificial Sequence
  Synthetic
atggcttgcaactgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaagatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacagcggccgcatccggagggcccacaatcaagccctgtcctccatgcaaatgcccagcacctaacctcttgggtggaggatccggccagccacccaaactcctcatctatgatgcatccaatctagtttctgggattccacccaggtttagtggcagtgggtctgggacagacttcaccctcaacatccatcctgtggagaaggtggatgctgcaacctatcactgtcagcaaagtactgaagatccgtggacgttcggtggagggaccaagctggaaataaaatctcccggttcaggaggaggtggctccggtggaggagggtctggcggaggaggctcaagcggagagctcgtgctcacccagtctccagcttctttggctgtgtctctagggcagagggccaccatctcctgcaaggccagccaaagtgttgattatgatggtgatagttatttgaactggtaccaacagattccaggaagcggctctggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctccagggagtggcggcggcggatccggaggaggcgggtcaggtggcggcggctcatctgggcaagtgcagctcctggagtctggggctgagctggtgaggcctgggtcctcagtgaagatttcctgcaaggcttctggctatgcattcagtagctactggatgaactgggtgaagcagaggggtggaggcggttcccatcaccatcaccatcactgatgaagatctcaattggaattctga

SEQ ID NO: 32 - protein, 562 aa, Artificial Sequence
  Synthetic
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTAAASGGPTIKPCPPCKCPAPNLLGGGSGQPPKLLIYDASNLVSGIPPRFSGSGSGTDFTLNIHPVEKVDAATYHCQQSTEDPWTFGGGTKLEIKSPGSGGGGSGGGGSGGGGSSGELVLTQSPASLAVSLGQRATISCKASQSVDYDGDSYLNWYQQIPGSGSGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRPGSSVKISCKASGYAFSSYWMNWVKQRGGGGSHHHHHH

SEQ ID NO: 33 - DNA, 888 bp, Artificial Sequence
  Synthetic
cagccacccaaactcctcatctatgatgcatccaatctagtttctgggattccacccaggtttagtggcagtgggtctgggacagacttcaccctcaacatccatcctgtggagaaggtggatgctgcaacctatcactgtcagcaaagtactgaagatccgtggacgttcggtggagggaccaagctggaaataaaatctcccggttcaggaggaggtggctccggtggaggagggtctggcggaggaggctcaagcggagagctcgtgctcacccagtctccagcttctttggctgtgtctctagggcagagggccaccatctcctgcaaggccagccaaagtgttgattatgatggtgatagttatttgaactggtaccaacagattccaggaagcggctctggacagggtcttgagtggattggacagatttggcctggagatggtgatactaactacaatggaaagttcaagggtaaagccactctgactgcagacgaatcctccagcacagcctacatgcaactcagcagcctacgatctgaggactctgcggtctattcttgtgcaagacgggagactacgacggtaggccgttattactatgctatggactactggggccaagggaccacggtcacctctccagggagtggcggcggcggatccggaggaggcgggtcaggtggcggcggctcatctgggcaagtgcagctcctggagtctggggctgagctggtgaggcctgggtcctcagtgaagatttcctgcaaggcttctggctatgcattcagtagctactggatgaactgggtgaagcagaggggtggaggcggttcccatcaccatcaccatcactgatgaagatctcaattggaattctga

SEQ ID NO: 34 - protein, 287 aa, Artificial Sequence
  Synthetic
QPPKLLIYDASNLVSGIPPRFSGSGSGTDFTLNIHPVEKVDAATYHCQQSTEDPWTFGGGTKLEIKSPGSGGGGSGGGGSGGGGSSGELVLTQSPASLAVSLGQRATISCKASQSVDYDGDSYLNWYQQIPGSGSGQGLEWIGQIWPGDGDTNYNGKFKGKATLTADESSSTAYMQLSSLRSEDSAVYSCARRETTTVGRYYYAMDYWGQGTTVTSPGSGGGGSGGGGSGGGGSSGQVQLLESGAELVRPGSSVKISCKASGYAFSSYWMNWVKQRGGGGSHHHHHH

SEQ ID NO: 35 - DNA, 60 bp, Artificial Sequence
  Synthetic
cctggatccggtggaggcggatcaggtggcggtggaagtggaggtggtggatcttccgga

SEQ ID NO: 36 - protein, 20 aa, Artificial Sequence
  Synthetic
PGSGGGGSGGGGSGGGGSSG

SEQ ID NO: 37 - DNA, 57 bp, Artificial Sequence
  Synthetic
ggatccggtggaggcggatcaggtggcggtggaagtggaggtggtggatcttccgga

SEQ ID NO: 38 - protein, 19 aa, Artificial Sequence
  Synthetic
GSGGGGSGGGGSGGGGSSG

SEQ ID NO: 39 - DNA, 75 bp, Artificial Sequence
  Synthetic
cctggatccggtggaggcggatcaggtggcggtggaagtggaggtggtggatctggcggaggcggatcttccgga

SEQ ID NO: 40 - protein, 25 aa, Artificial Sequence
  Synthetic
PGSGGGGSGGGGSGGGGSGGGGSSG

SEQ ID NO: 41 - DNA, 72 bp, Artificial Sequence
  Synthetic
ggatccggtggaggcggatcaggtggcggtggaagtggaggtggtggatctggcggaggcggatcttccgga

SEQ ID NO: 42 - protein, 24 aa, Artificial Sequence
  Synthetic
GSGGGGSGGGGSGGGGSGGGGSSG

SEQ ID NO: 43 - DNA, 90 bp, Artificial Sequence
  Synthetic
cctggatccggtggaggcggatcaggtggcggtggaagtggaggtggtggatctggcggaggcggatctggaggtggaggctcttccgga

SEQ ID NO: 44 - protein, 30 aa, Artificial Sequence
  Synthetic
PGSGGGGSGGGGSGGGGSGGGGSGGGGSSG

SEQ ID NO: 45 - DNA, 87 bp, Artificial Sequence
  Synthetic
ggatccggtggaggcggatcaggtggcggtggaagtggaggtggtggatctggcggaggcggatctggaggtggaggctcttccgga

SEQ ID NO: 46 - protein, 29 aa, Artificial Sequence
  Synthetic
GSGGGGSGGGGSGGGGSGGGGSGGGGSSG

SEQ ID NO: 47 - DNA, 18 bp, Artificial Sequence
  Synthetic
ggaggttccgcggccgca

SEQ ID NO: 48 - protein, 6 aa, Artificial Sequence
  Synthetic
GGSAAA

SEQ ID NO: 49 - DNA, 15 bp, Artificial Sequence
  Synthetic
ggtggaggcggttcc

SEQ ID NO: 50 - protein, 5 aa, Artificial Sequence
  Synthetic
GGGGS

SEQ ID NO: 51 - DNA, 9 bp, Artificial Sequence
  Synthetic
gcggccgca

SEQ ID NO: 52 - protein, 3 aa, Artificial Sequence
  Synthetic
AAA

SEQ ID NO: 53 - DNA, 12 bp, Artificial Sequence
  Synthetic
cctggatccggt

SEQ ID NO: 54 - protein, 4 aa, Artificial Sequence
  Synthetic
PGSG

SEQ ID NO: 55 - DNA, 18 bp, Artificial Sequence
  Synthetic
ggtcctggatccggtgga

SEQ ID NO: 56 - protein, 6 aa, Artificial Sequence
  Synthetic
GPGSGG

SEQ ID NO: 57 - DNA, 24 bp, Artificial Sequence
  Synthetic
ggtggacctggatccggtggaggc

SEQ ID NO: 58 - protein, 8 aa, Artificial Sequence
  Synthetic
GGPGSGGG

(19) United States
(12) Patent Application Publication
(10) Pub. No.: US 2023/0272071 A1
(43) Pub. Date: Aug. 31, 2023

(54) MULTIFUNCTIONAL IMMUNOGLOBULIN-FOLD POLYPEPTIDES FROM ALTERNATIVE TRANSLATIONAL INITIATION AND TERMINATION

(71) Applicants: Michael S. KUHNS, Tucson, AZ (US); Mark S. LEE, Tucson, AZ (US); Deepta BHATTACHARYA, Tucson, AZ (US); ARIZONA BOARD OF REGENTS ON BEHALF OF THE UNIVERSITY OF ARIZONA, Tucson, AZ (US)

(72) Inventors: Michael S. KUHNS, Tucson, AZ (US); Mark S. LEE, Tucson, AZ (US); Deepta BHATTACHARYA, Tucson, AZ (US)

(21) Appl. No.: 18/006,698

(22) PCT Filed: Jul. 26, 2021
(86) PCT No.: PCT/US2021/043126

§ 371 Date: Jan. 24, 2023

Related U.S. Application Data
(60) Provisional application No. 63/056,785, filed on Jul. 27, 2020.

(51) Int. Cl. C07K 16/28; C12N 15/63

(52) U.S. Cl. CPC C07K 16/2803; C12N 15/63; C07K 2317/35; C07K 2317/622

(57) ABSTRACT

Described herein are nucleotide and polypeptide sequences of alternative immunoglobulin domains, engineered to create non-native N′- and C′-termini in the loop regions of the three-dimensional β-sheet structure of the domain, for use as fusion partners with additional polypeptide sequences. Such polypeptides can be used as reagents, research tools, or therapeutics.

### CROSS-REFERENCE TO RELATED APPLICATIONS

This application claims priority to U.S. Provisional Patent Application No. 63/056,785, filed on 27 Jul. 2020, and is hereby incorporated by reference herein in its entirety.

### REFERENCE TO SEQUENCE LISTING

This application is filed with a Computer Readable Form of a Sequence Listing in accord with 37 C.F.R. § 1.821(c). The text file submitted by EFS, “212443-9007-WO01_sequence_listing_20 Jul. 2021_ST25.txt,” was created on 20 Jul. 2021, contains 58 sequences, has a file size of 85.7 Kbytes, and is hereby incorporated by reference herein in its entirety.

### TECHNICAL FIELD

Described herein are nucleotide and polypeptide sequences of alternative immunoglobulin domains, engineered to create non-native N′- and C′-termini in the loop regions of the three-dimensional β-sheet structure of the domain, for use as fusion partners with additional polypeptide sequences. Such polypeptides can be used as reagents, research tools, or therapeutics.

### BACKGROUND

Immunoglobulin (Ig) domains are essential building blocks for the synthesis of a variety of different proteins, including immunoglobulins (antibodies), MHC, and cell receptors, e.g., T-cell receptors (TCRs), CD2, CD4, CD8, receptors, CD80, CTLA-4, PD1, and PDL1, inter alia. See e.g., Williams and Barclay, Ann. Rev. Immunol. 6:381-405 (1988); Bork et al, J. Mol. Biol. 242: 309-320 (1994); Harpaz and Chothia, J. Mol. Biol. 238: 528-539 (1994); Clarke et al., Structure 7:1145-1153 (1999); Halaby et al., Prot. Eng. 12(7): 563-571 (1999); and Abhinandan and Martin, J. Mol. Biol. 369:852-862 (2007), each of which are incorporated by reference herein for such teachings. Immunoglobulins (antibodies) are prime examples of proteins built with Ig domains. Antibodies are heterodimers comprising two heavy chains and two light chain molecules linked by disulfide linkages. Each heavy and light chain consists of a constant region that is highly conserved and a variable region that confers the binding specificity. Each heavy and light chain consists of a series of linked Ig domains with tertiary subdomains consisting of β-sheets that form a β-sandwich structural motif. The individual p-strands forming the β-sheets are connected by loop regions.

While Ig domain-containing proteins have proven useful as reagents, research tools, or therapeutics, there are restrictions put on the manner in which they can be used because translation occurs from the N- to C-terminus. What is needed is a method to construct novel immunoglobulin domains that allow for inserting linkers or other protein domains within the loop regions of immunoglobulin domains to create novel proteins with multiple functions such as reagents, research tools, or therapeutics.

### SUMMARY

One embodiment described herein is a nucleotide sequence encoding a polypeptide, where the polypeptide comprises one or more immunoglobulin domains comprising fusion of the wild type N- and C-termini or an optional linker joining the wild type N- and C-termini and a scission within one of the loop regions yielding novel N′- and C′-termini. In one aspect, the immunoglobulin domain comprises an immunoglobulin domain from an immunoglobulin, Fab, Fv, T cell receptor (TCR), CD80, CTLA-4, PD1, PDL1, MHC molecules or other immunoglobulin domain containing proteins. In another aspect, the immunoglobulin domain comprises a heavy chain variable domain or a light chain variable domain. In another aspect, one or both of the N′- and C′-termini are fused with one or more additional polypeptides. In another aspect, the additional polypeptide comprises an immunoglobulin domain, Fab, Fv, ScFV, cell receptor, pMHC, costimulatory molecule, cytokine, or another polypeptide domain. In another aspect, the additional polypeptide comprises an immunoglobulin hinge region. In another aspect, the additional polypeptide domain comprises one or more of: CD80, CD86, ICAM-1, PD-L1/L2, B7H1, B7H2, CD40, CD40L, CD47, CD48, CD58, 4-1BBL, OX40L, TIM-1, TIM-4, CD80:PD-L1 heterodimer, calreticulin, a peptide that is at least 90% identical to CD80, CD86, ICAM-1, PD-L1/L2, B7H1, B7H2, CD40, CD40L, CD47, CD48, CD58, 4-1BBL, OX40L, TIM-1, TIM-4, CD80:PD-L1 heterodimer, calreticulin, fragments thereof, or combinations thereof; cytokines: IFNα, IFNβ, IFNγ, IL-1, IL-1a, IL-2, IL-4, IL-6, IL-7, IL-10, IL-12, IL-15, IL-21, IL-23, TNF, TNFα, TGFβ, GM-CSF, CSF-1, a peptide that is at least 90% identical to IFNα, IFNβ, IFNγ, IL-1, IL-1a, IL-2, IL-4, IL-6, IL-7, IL-10, IL-12, IL-15, IL-21, IL-23, TNF, TNFα, TGFβ, GM-CSF, CSF-1, fragments thereof, or combinations thereof; MHC alleles: MHC molecule comprises HLA-A, HLA-B, HLA-C, P2-microglobulin, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKP, a peptide that is at least 90% identical to HLA-A, HLA-B, HLA-C, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ, fragments thereof, or combinations thereof; or TCR molecules: TRAC, TRBC1, TRBC2, TRDC, TRGC1, TRGC2, TCRA, TCB1, TCB2, TCC1, TCC2, TCC3, TCC4, a peptide that is at least 90% identical to TRAC, TRBC1, TRBC2, TRDC, TRGC1, TRGC2, TCRA, TCB1, TCB2, TCC1, TCC2, TCC3, TCC4, fragments thereof, or combinations thereof. In another aspect, the optional linker comprises a polypeptide linker or a chemical linker. In another aspect, the optional linker comprises a polypeptide selected from one or more of a poly glycine linker, poly alanine linker, poly glycine-alanine linker, poly glycine-serine linker, or poly glycine-serine-proline linker. In another aspect, the optional linker comprises a polypeptide having 85% to 99% identity to one or more of SEQ ID NO: 36, 38, 40, 42, 44, 46, 48, 50, 52, 54, 56, or 58. In another aspect, the optional linker is a polypeptide selected from one or more of SEQ ID NO: 36, 38, 40, 42, 44, 46, 48, 50, 52, 54, 56, or 58. In another aspect, the loop region where scission occurs comprises one or more of the loops connecting adjacent β-strands comprising A-B, B-C, C-C′, C′-C″, C-D, C′-D, C″-D, D-E, E-F, F-G, or other loop-linkages that eliminate one or more intervening β-strands. In another aspect, the loop region where scission occurs comprises the C′-C″ or A-B loop. In another aspect, the nucleotide sequence has 85% to 99% identity to SEQ ID NO: 1, 3, 5, 7, 9, 11, 13, 15, 17, 19, 21, 23, 25, 27, 29, 31, or 33. In another aspect, the nucleotide sequence is selected from SEQ ID NO: 1, 3, 5, 7, 9, 11, 13, 15, 17, 19, 21, 23, 25, 27, 29, 31, or 33.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment is a polypeptide encoded by a nucleotide sequence described herein. In one aspect, the polypeptide has 85% to 99% identity to SEQ ID NO: 2, 4, 6, 8, 10, 12, 14, 16, 18, 20, 22, 24, 26, 28, 30, 32, or 34. In another aspect, the polypeptide is selected from SEQ ID NO: 2, 4, 6, 8, 10, 12, 14, 16, 18, 20, 22, 24, 26, 28, 30, 32, or 34.

Another embodiment described herein is a bivalent polypeptide complex comprising a dimer of the polypeptides of SEQ ID NO: 32 or 34 covalently linked via one or more disulfide bonds.

Another embodiment described herein is a single chain variable fragment (scFv) polypeptide comprising SEQ ID NO: 34.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the means comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by the method or the means described herein Another embodiment described herein is a method of treatment comprising administering an effective amount of polypeptide encoded by one or more of the nucleotide sequences described herein a subject in need thereof.

Another embodiment described herein is the use of an effective amount of a polypeptide encoded by one or more of the nucleotide sequences described herein for the treatment of a disease or disorder comprising a administering an effective amount of polypeptide encoded by the nucleotide sequences to a subject in need thereof.

Another embodiment described herein is a research tool comprising a polypeptide encoded by a nucleotide sequence described herein.

Another embodiment described herein is an immunochemical reagent comprising a polypeptide encoded by a nucleotide sequence described herein.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the three-dimensional structure of an exemplary Fab fragment of an immunoglobulin (Ig). The constant and variable regions of the light (magenta) and heavy (cyan) chains are labeled to highlight that each region is composed of pairs of immunoglobulin domains, with one Ig domain contributed from each chain.

FIG. 2A shows the light and heavy chains of the Fab fragment separated to highlight the tertiary arrangement of the individual constant and variable region Ig domains. The top and bottom β-sheets of the Ig domains are indicated.

FIG. 2B shows the tertiary arrangement of the β-sheets of the Fab fragment heavy and light chains. Individual β-strands and inter-strand loops are indicated.

FIG. 3A shows a schematic diagram of the arrangement of immunoglobulin β-sheets. The N-terminus is on p-strand A and the C-terminus is on p-strand G.

FIG. 3B shows a schematic diagram of the arrangement of AltIg β-sheets where a linker is placed between p-strand A and p-strand G, and the loop region between β-strands C and C′ is severed. The N′-terminus is now on p-strand C′ and the C′-terminus is on p-strand C.

FIG. 4A shows a comparison of the three-dimensional structure of a wild-type immunoglobulin (Ig) domain with a model of an AltIg Domain.

FIG. 4B shows alternative views of the three-dimensional model structures of an AltIg Domain.

FIG. 5A shows a comparison of the quarternary three-dimensional model structure of an AltIg Fv with a wild-type variable fragment (Fv). Each Fv domain consists of both a heavy chain and a light chain domain.

FIG. 5B shows a close-up view of an AltIg Fv with the new N′- and C′-termini.

FIG. 6 shows the results of a flow-based fluorophore-linked immunosorbant assay (FFLISA) for AltIg construct shown in FIG. 5B having a myc and HA tag on the N′-terminus of the light chain or heavy chain, respectively, as well as a 6×His tag at the C′-terminus. Culture media (negative control) or supernatants from M12 cells expressing AltIgs were incubated with polystyrene beads coated with anti-His tag antibodies, stained with fluorophore conjugated antibodies against the HA or Myc tag, and analyzed for fluorescent intensity by flow cytometry. These results indicate that AltIgs are secreted from cells as soluble molecules. The construct is shown below the results.

FIG. 7A shows examples of the use of AltIg molecules in the generation of Biomimetic Stimulators (BMiMS). BMiMS are composed of AltIg Fv regions from an antibody that binds a cell surface antigen (e.g., a tumor antigen such as CD19 on B cell lymphomas) fused directly to molecules that are integral to the activation of T cells (e.g., pMHCII, CD80, CD86, and ICAM-1). The illustration depicts how the use of the AltIg engineering for direct fusion to the activation molecules situates the stimulatory molecules in the proper spatial orientation with respect to the target cell membrane for recognition by a T cell (e.g., the N-terminal region of pMHCII is pointing away from the tumor cell surface and towards a T cell). Such an orientation could not be achieved with a N- to C-terminal fusion of a stimulatory molecule to a native Fv region.

FIG. 7B shows the results of a flow-based fluorophore-linked immunosorbant assay (FFLISA) for AltIg-based BMiMS. Culture media or supernatant from M12 cells expressing BMiMS were incubated with polystyrene beads coated with anti-His tag antibodies (captures His tag at the C-terminus of the AltIg), stained with fluorophore conjugated antibodies against the indicated immune molecules, and analyzed for fluorescent intensity by flow cytometry. These results indicate that BMiMS are secreted from cells as soluble molecules.

FIG. 8 shows the results of flow cytometry analysis of Ramos cells stained with BMiMS. Ramos cells were incubated with culture media (negative control) or supernatant from M12 cells expressing BMiMS, then stained with fluorophore conjugated antibodies against the indicated immune molecules and analyzed by flow cytometry. These results show that BMiMS AtlIgs bind to B-cell lymphomas (Ramos cells) in vitro.

FIG. 9A shows a cartoon illustration of the experimental design for stimulating naive CD4+ T cells with BMiMS.

FIG. 9B shows results of IL-2 ELISA from naive 5c.c7+CD4+ T cells after 18 hrs culture in plates coated with rhuCD19 and BMiMS. Bars represent the means of triplicate wells ±SEM.

FIG. 10A shows a schematic diagram of the arrangement of immunoglobulin β-sheets. The N-terminus is on β-strand A and the C-terminus is on β-strand G.

FIG. 10B shows a schematic diagram of the arrangement of AltIg β-sheets where a linker is placed between β-strand A and β-strand G, and the loop region between β-strands A and B is severed. The N′-terminus is now on β-strand B and the C′-terminus is on β-strand A.

FIG. 11 shows the results of a flow-based fluorophore-linked immunosorbant assay (FFLISA) for the AltIg construct shown in FIG. 10B having a myc and HA tag on the N′-terminus of the light chain or heavy chain, respectively, as well as a 6×His tag at the C′-terminus FIG. 12 shows a model of an AltIg-based single chain (sc) fragment of variable (Fv) region, i.e., an AltIg-scFv.

FIG. 13 shows a model of an AltIg-scFv with an IgG2a hinge region fused to the AltIg LC N-terminus.

FIG. 14 shows a model of an AltIg-scFv with an IgG2a hinge region fused to the AltIg LC N-terminus for making bivalent AltIg-scFvs.

FIG. 15 shows a cartoon of a BMiMS composed of a bivalent anti-hCD19 AltIg-scFv with an IgG2a hinge region and CD80. The illustration depicts how the bivalent BMiMS situates the molecules in the proper spatial orientation with respect to the target cell membrane for recognition.

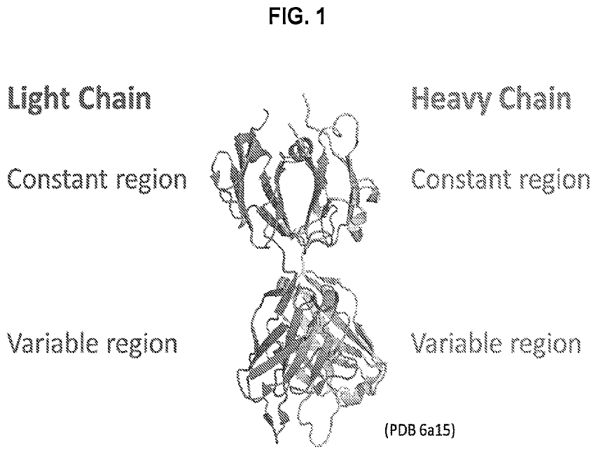

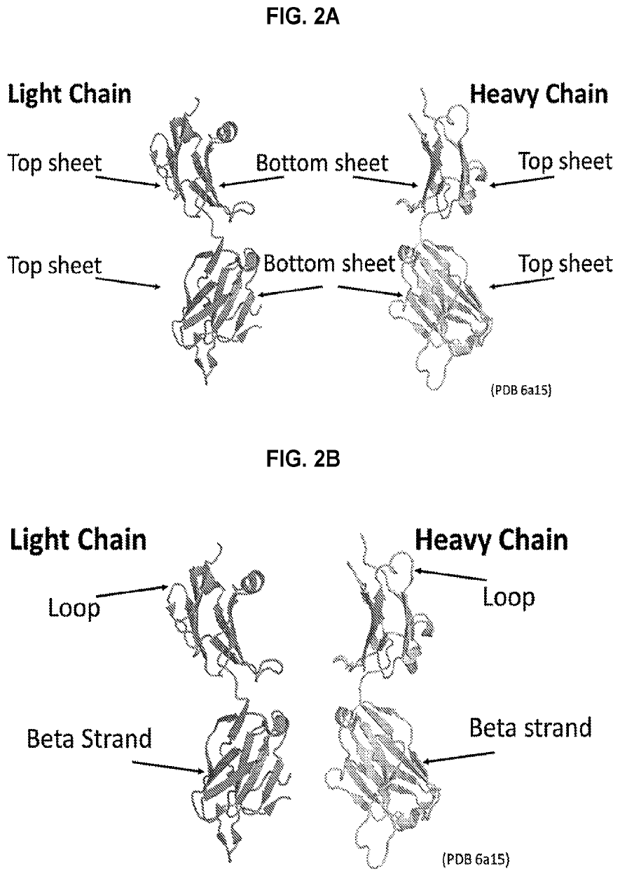

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polypeptide,” “polynucleotide,” and “vector” have their common meanings as would be understood by a biochemist of ordinary skill in the art. Standard single letter nucleotides (A, C, G, or T) and standard single letter amino acids (A, C, D, E, F, G, H, I, K, L, M, N, P, Q, R, S, T, V, W, or R) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

Described herein are nucleotides encoding polypeptides and polypeptides comprising Alternative Immunoglobulin Domains (“AltIgs”). By altering the position of the first (N-terminus) and last (C-terminus) amino acids of proteins that adopt an immunoglobulin (Ig) fold, and the insertion of linkers and other peptides, Alternative Immunoglobulins (“AltIgs”) can be constructed. One embodiment of an AltIg example is the fusion of an AltIg to another protein as a single polypeptide in order to achieve a different spatial relationship between the functional end of the AltIg and the second protein that could not be achieved if the native Ig were fused to the second protein in a conventional manner (i.e., with the N-termini of the second protein being fused to the C-termini of a native Ig). The AltIg design permits the positioning of the amino-terminal (N-) regions of the AltIg distal to the N-terminal region of the second protein, while the carboxy-terminal (C-) regions of both proteins would be proximal. Such constructs are important because protein translation naturally occurs from the N- to the C-terminus of a polypeptide, and this rule of nature creates engineering challenges for the design and synthesis of novel bifunctional polypeptides in which one side has a specific function (e.g., an antibody variable region (Fv) that binds target A) and the other portion has a distinct function (e.g., an Fv or other protein that binds target B). The reason why this can be problematic concerns the orientation of the functional ends of the two proteins fused together as one long polypeptide where the C-terminus of the first protein in linked to the N-terminus of the second protein. Linkers can be optionally added between the C termini and N-termini to adjust the spacing or permit flexibility. Specifically, if the functional sites of both the first and second protein are at their N-termini, such a design would put the functional site of the second protein in a suboptimal in-line orientation relative to the first. However, for many potential experimental tools or therapeutic agents, it may be more desirable to have the two functional N-termini of the two proteins at distal ends. The problem is that constructing such polypeptides by conventional means is impossible in a continuous polypeptide and would require other means of linking the protein (e.g., chemical linkers or engineered non-covalent protein interaction sites). The problem becomes even more challenging if the two proteins fused together consist of multiple subunits. For example, to engineer a bifunctional protein consisting of an antibody Fv that binds an epitope on a tumor cell on one end, and a pMHC molecule that could be detected by a T cell on the other end, there are two challenges. The first is that both the antigen-binding site on the Fv and the T cell recognition site on the pMHC are at the N-termini of both proteins. Therefore, if the pMHC was linked to the Fv in a natural polypeptide orientation, then when the Fv is bound to the tumor cell the pMHC would also be facing towards the tumor cell instead of outward for recognition by a T cell. The second problem is that both the Fvs and pMHC are heterodimers, making it more challenging to fuse as one polypeptide.

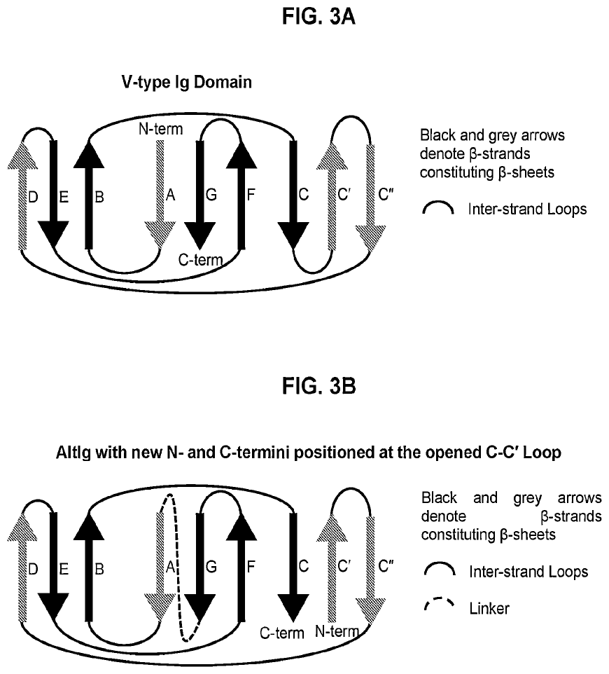

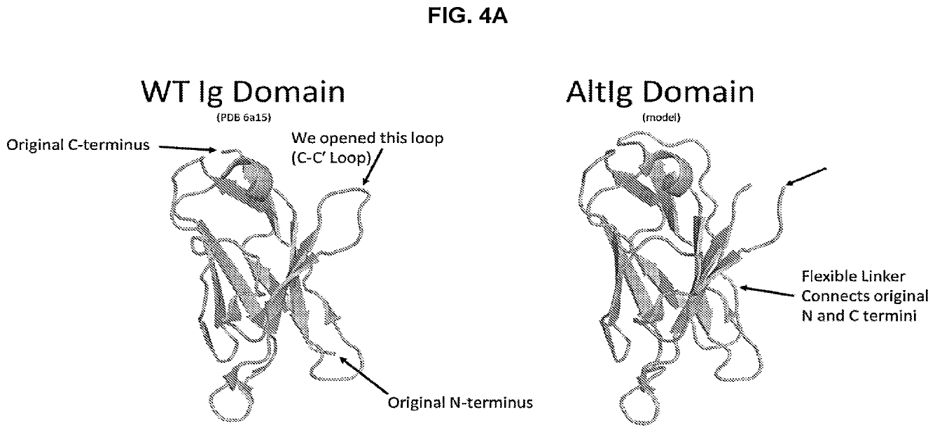

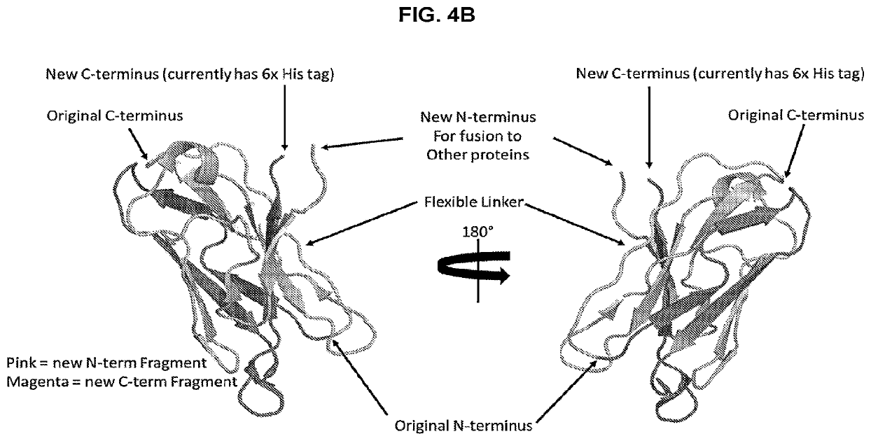

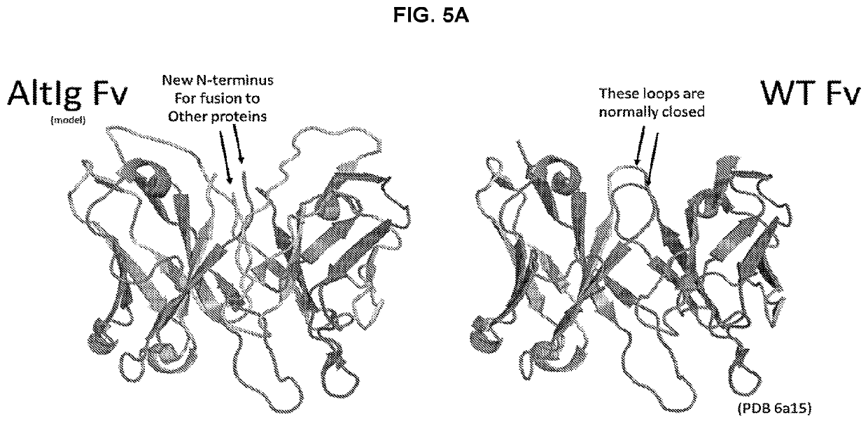

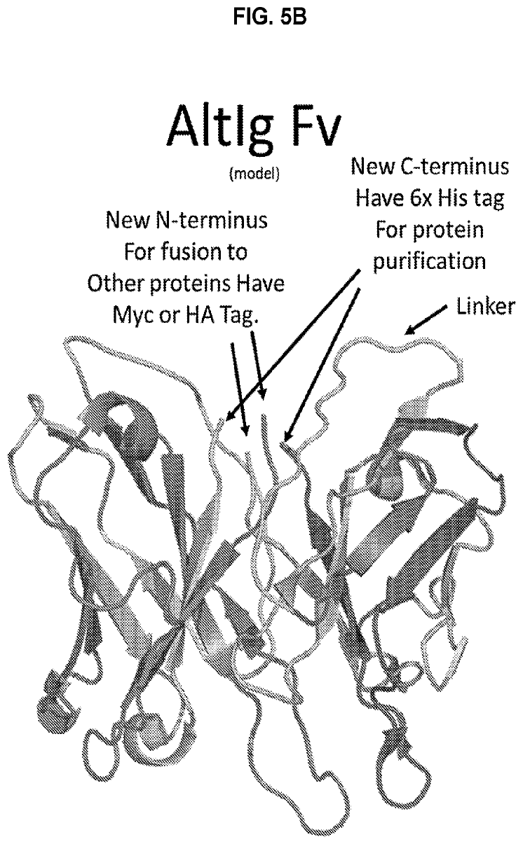

The constructs described herein change where translation of an Ig domain starts and stops and have a fusion of the former (native) N- and C-termini or a flexible linker connecting the former N- and C-termini to ensure continuity of the resultant polypeptide chain. See FIG. 3A-B. By making such alterations, the C-terminus of a desired protein can be translated as a continuous polypeptide with the novel N′-terminus of the AltIg domain to create a polypeptide where what are normally the N-terminal regions of each protein component (e.g., the AltIg and a conventional Fv or a pMHC) are distal to each other. Alternatively, the N-terminus of a desired protein can be translated as a continuous polypeptide from the C′-terminus of the AltIg to achieve a different orientation of each protein component.

Any protein containing an immunoglobulin domain can be used as the initial polypeptide domain for constructing an AltIg. Proteins containing Ig domains include but are not limited to those in the immunoglobulin superfamily (EMBL-EBI Family PF00047, clan CL0011; NCBI Conserved Protein Domain Family cd00096, which are incorporated by reference herein for such teachings) including antibodies or immunoglobulins (IgA, IgD, IgE, IgG, IgM); T-cell receptors (TRAC, TRBC1, TRBC2, TRDC, TRGC1, TRGC2, TCRA, TCB1, TCB2, TCC1, TCC2, TCC3, TCC4); Antigen presenting molecules (Class I MHC, Class II MHC, β-2 microglobulin, HLA-A, HLA-B, HLA-C, β2-microglobulin, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ); co-receptors (CD4, CD8, CD19); antigen receptor accessory molecules (CD3-γ, -δ, and -ε chains, CD79a and CD79b); co-stimulatory or inhibitory molecules (CD28, CD80, CD86, PD-1, PD-L1); killer-cell immunoglobulin-like receptors (KIR)); leukocyte immunoglobulin-like receptors (LILR); IgSF CAMs (NCAMs, ICAM-1, CD2 subset); cytokine receptors (Interleukin-1 receptor, Colony stimulating factor 1 receptor); Growth factor receptors (Platelet-derived growth factor receptor (PDGFR), Mast/stem cell growth factor receptor precursor (SCFR, c-kit, CD117 antigen)); receptor tyrosine kinases/phosphatases (tyrosine-protein kinase receptor Tie-1 precursor, Type IIa and Type IIb Receptor protein tyrosine phosphatases (RPTPs), including, but not limited to, PTPRM, PTPRK, PTPRU, PTPRD, PTPRF); Ig binding receptors (polymeric immunoglobulin receptor (PIGR), Some Fc receptors); cytoskeleton proteins (myotilin, myopalladin, palladin, Titin, Obscurin, MYOM1, MYOM2); CD147; CD90; CD7; Butyrophilins (Btn), and other proteins. See e.g., Chothia and Lesk, J. Mol. Biol. 196(4):901-917 (1987); Williams and Barclay, Annu. Rev. Immunol. 6:381-405 (1988); Bork et al., J. Mol. Biol. 242(4):309-320 (1994); Harpaz and Chothia, J. Mol. Biol. 238: 528-539 (1994); BrQmmendorf and Rathjen, Protein Profile 2(9):963-1108 (1995); Halaby and Mornon, J. Mol. Evol. 46(4): 389-400 (1998); Chothia and Kister, J. Mol. Biol. 278(2):457-479 (1998); Litman et al., Annu. Rev. Immunol. 17:109-147 (1999); Clarke et al., Structure 7:1145-1153 (1999); Halaby et al., Protein Eng. 12(7):563-571 (1999); Zuccotti et al., Acta Crystallogr. D Biol. Crystallogr. 59 (Pt 7):1270-1272 (2003); and Abhinandan and Martin, J. Mol. Biol. 369:852-862 (2007), each of which is incorporated by reference herein for such teachings. Species of organisms having any of the above described immunoglobulin domains include but are not limited to human, chimpanzee, gorilla, orangutan, other non-human primates, mouse, rat, rabbit, goat, horse, camel, pig, cow, sheep, dog, cat, and other mammals.

Numerous proteins, subdomains, or peptides can be fused to the AltIg at the new N′- or C′-termini. Any of the foregoing immunoglobulin domain proteins discussed herein can be fused to make AltIgs with one or more immunoglobulin domains, each with a particular specificity. In addition, the AltIg can be fused with immunoglobulin domains, Fab, Fv, ScFV, cell receptor, pMHC, costimulatory molecule, cytokine, or other polypeptide domains. Exemplary proteins include CD80, CD86, ICAM-1, PD-L1/L2, B7H1, B7H2, CD40, CD40L, CD47, CD48, CD58, 4-1BBL, OX40L, TIM-1, TIM-4, CD80:PD-L1 heterodimer, calreticulin; cytokines: IFNα, IFNβ, IFNγ, IL-1, IL-1a, IL-2, IL-4, IL-6, IL-7, IL-10, IL-12, IL-15, IL-21, IL-23, TNF, TNFα, TGFβ, GM-CSF, CSF-1; MHC alleles: MHC molecule comprises HLA-A, HLA-B, HLA-C, β2-microglobulin, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ, a peptide that is at least 90% identical to HLA-A, HLA-B, HLA-C, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ, fragments thereof, or combinations thereof.

One intended use of the AltIg technology is to produce AltIg Fvs that bind tumor antigens and are fused to either pMHC of a known antigenicity (e.g., recognized by anti-CMV T cells), costimulatory molecules (e.g., CD80), adhesion molecules (e.g., ICAM-1), and cytokines (e.g., IL-12). Because Fvs are heterodimers composed of a light chain (LC) Ig fold and a heavy chain (HC) Ig fold, AltIgs can be made out of both the LC and HC for a given Fv. Each could be fused to a subunit of another heterodimer (e.g., natural Fv subunits or the α and β subunits of pMHCII) to generate a reagent with an AltIg Fv on one end and a pMHC, costimulatory molecule, or adhesion molecule, or cytokine on the other. As used herein, such constructs are termed “Biomimetic Stimulators” (“BMiMS”). Tumor cells can be contacted with BMiMS that comprise four critical components for T cell activation—antigen (pMHC), costimulation, adhesion molecules, and signaling (via cytokines)—to render the tumor cells susceptible to targeting by T cell populations that are present in most individuals due to infection with common viruses such as hCMV, flu, vaccinia, etc. Numerous other applications are envisioned.

Another embodiment described herein is a therapeutic compound made with AltIgs. In one embodiment, the therapeutic compound comprises a BMiMS. In another embodiment, reagents can be constructed to target tumor cells with the critical signals that are required to make the tumor cell susceptible to phagocytosis by innate immune cells by fusing an anti-tumor AltIg Fv to calreticulin as an “eat me” signal. Other embodiments include anti-drug Fvs for delivering drugs to specific cell types or tissues.

One embodiment described herein is a nucleotide sequence encoding a polypeptide, where the polypeptide comprises one or more immunoglobulin domains comprising a fusion of or an optional linker joining the wild type N- and C-termini and a scission within one of the loop regions yielding novel N′- and C′-termini. In one aspect, the immunoglobulin domain comprises an immunoglobulin domain from an immunoglobulin, Fab, Fv, T cell receptor (TCR), CD80, CTLA-4, PD1, PDL1, MHC molecules or other immunoglobulin domain containing proteins. In another aspect, the immunoglobulin domain comprises a heavy chain variable domain or a light chain variable domain. In another aspect, one or both of the N′- and C′-termini are fused with one or more additional polypeptides. In another aspect, the additional polypeptide comprises an immunoglobulin domain, Fab, Fv, ScFV, cell receptor, pMHC, cytokine, costimulatory molecule, or other polypeptide domain. In another aspect, the additional polypeptide domain In another aspect, the additional polypeptide domain comprises one or more of: CD80, CD86, ICAM-1, PD-L1/L2, B7H1, B7H2, CD40, CD40L, CD47, CD48, CD58, 4-1BBL, OX40L, TIM-1, TIM-4, CD80:PD-L1 heterodimer, calreticulin, a peptide that is at least 90% identical to CD80, CD86, ICAM-1, PD-L1/L2, B7H1, B7H2, CD40, CD40L, CD47, CD48, CD58, 4-1BBL, OX40L, TIM-1, TIM-4, CD80:PD-L1 heterodimer, calreticulin, fragments thereof, or combinations thereof; cytokines: IFNα, IFNβ, IFNγ, IL-1, IL-1a, IL-2, IL-4, IL-6, IL-7, IL-10, IL-12, IL-15, IL-21, IL-23, TNF, TNFα, TGFβ, GM-CSF, CSF-1, a peptide that is at least 90% identical to IFNα, IFNβ, IFNγ, IL-1, IL-1a, IL-2, IL-4, IL-6, IL-7, IL-10, IL-12, IL-15, IL-21, IL-23, TNF, TNFα, TGFβ, GM-CSF, CSF-1, fragments thereof, or combinations thereof; MHC alleles: MHC molecule comprises HLA-A, HLA-B, HLA-C, β2-microglobulin, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ, a peptide that is at least 90% identical to HLA-A, HLA-B, HLA-C, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ, fragments thereof, or combinations thereof; or TCR molecules: TRAC, TRBC1, TRBC2, TRDC, TRGC1, TRGC2, TCRA, TCB1, TCB2, TCC1, TCC2, TCC3, TCC4, a peptide that is at least 90% identical to TRAC, TRBC1, TRBC2, TRDC, TRGC1, TRGC2, TCRA, TCB1, TCB2, TCC1, TCC2, TCC3, TCC4, fragments thereof, or combinations thereof. In another aspect, the linker comprises a polypeptide linker or a chemical linker. In another aspect, if used, the linker comprises a polypeptide selected from one or more of a poly glycine linker, poly alanine linker, poly glycine-alanine linker, poly glycine-serine linker, or poly glycine-serine-proline linker. In another aspect, the linker comprises a polypeptide having 85% to 99% identity to one or more of SEQ ID NO: 36, 38, 40, 42, 44, 46, 48, 50, 52, 54, 56, or 58. In another aspect, the linker is a polypeptide selected from one or more of SEQ ID NO: 36, 38, 40, 42, 44, 46, 48, 50, 52, 54, 56, or 58. In another aspect, the loop region where scission occurs comprises one or more of the immunoglobulin β-sheet loops A-B, B-C, C-C′, C′-C″, C-D, C′-D, C″-D, D-E, E-F, or F-G, or other loop-linkages that eliminate one or more intervening β-strands. In another aspect, the loop region where scission occurs comprises one or more of the immunoglobulin β-sheet loops A-B, B-C, C-C′, C′-C″, C-D, C′-D, C″-D, D-E, E-F, or F-G. In another aspect, the loop region comprises the C′-C″ loop. In another aspect, the nucleotide sequence has 85% to 99% identity to SEQ ID NO: 1, 3, 5, 7, 9, 11, 13, 15, 17, 19, 21, 23, 25, 27, 29, 31, or 33. In another aspect, the nucleotide sequence is selected from SEQ ID NO: 1, 3, 5, 7, 9, 11, 13, 15, 17, 19, 21, 23, 25, 27, 29, 31, or 33.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment is a polypeptide encoded by a nucleotide sequence described herein. In one aspect, the polypeptide has 85% to 99% identity to SEQ ID NO: 2, 4, 6, 8, 10, 12, 14, 16, 18, 20, 22, 24, 26, 28, 30, 32, or 34. In another aspect, the polypeptide is selected from SEQ ID NO: 2, 4, 6, 8, 10, 12, 14, 16, 18, 20, 22, 24, 26, 28, 30, 32, or 34.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by the method or the means described herein Another embodiment described herein is a method of treatment comprising administering an effective amount of polypeptide encoded by one or more of the nucleotide sequences described herein a subject in need thereof.

Another embodiment described herein is the use of an effective amount of a polypeptide encoded by one or more of the nucleotide sequences described herein for the treatment of a disease or disorder comprising a administering an effective amount of polypeptide encoded by the nucleotide sequences to a subject in need thereof.

Another embodiment described herein is a research tool comprising a polypeptide encoded by a nucleotide sequence described herein.

Another embodiment described herein is an immunochemical reagent comprising a polypeptide encoded by a nucleotide sequence described herein.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% identical to (a) nucleotide sequences, or degenerate, homologous, or codon-optimized variants thereof, encoding polypeptides having the amino acid sequences in SEQ ID NO: 1, 3, 5, 7, 9, 11, 13, 15, 17, 19, 21, 23, 25, 27, 29, 31, or 33; (b) nucleotide sequences, or degenerate, homologous, or codon-optimized variants thereof, encoding polypeptides having the amino acid sequences in SEQ ID NO: 2, 4, 6, 8, 10, 12, 14, 16, 18, 20, 22, 24, 26, 28, 30, 32, or 34; and (c) nucleotide sequences capable of hybridizing to the complement of any of the nucleotide sequences in (a) or (b) above and capable of expressing functional polypeptides of amino acid sequences in SEQ ID NO: 2, 4, 6, 8, 10, 12, 14, 16, 18, 20, 22, 24, 26, 28, 30, 32, or 34.

By a polynucleotide having a nucleotide sequence at least, for example, 90-99% “identical” to a reference nucleotide sequence encoding AltIg is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding the AltIg.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2: 4 82-489 (1981). This algorithm can be extended to use with peptide sequences using the scoring matrix developed by Dayhoff, Atlas of Protein Sequences and Structure, M. O. Dayhoff ed., 5 suppl. 3: 353-358, National Biomedical Research Foundation, Washington, D.C., USA, and normalized by Gribskov, Nucl. Acids Res. 14(6): 6745-6763 (1986). An implementation of this algorithm for nucleic acid and peptide sequences is provided by the Genetics Computer Group (Madison, Wis.) in their BESTFIT utility application.

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, or 99% identical to the nucleic acid sequence shown in SEQ ID NO: 1, 3, 5, 7, 9, 11, 13, 15, 17, 19, 21, 23, 25, 27, 29, 31, or 33, or degenerate, homologous, or codon-optimized variants thereof, will encode an AltIg.

The polynucleotides described herein include those encoding mutations, variations, substitutions, additions, deletions, and particular examples of the polypeptides described herein. For example, guidance concerning how to make phenotypically silent amino acid substitutions is provided in Bowie, J. U. et al., “Deciphering the Message in Protein Sequences: Tolerance to Amino Acid Substitutions,” Science 247: 1306-1310 (1990), wherein the authors indicate that proteins are surprisingly tolerant of amino acid substitutions.

Thus, fragments, derivatives, or analogs of the polypeptides of SEQ ID NO: 2, 4, 6, 8, 10, 12, 14, 16, 18, 20, or 22 can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an IgG Fc fusion region peptide or leader or secretory sequence or a sequence which is employed for purification of the mature polypeptide or a proprotein sequence. Such fragments, derivatives, and analogs are deemed to be within the scope of those skilled in the art from the teachings herein.

In addition, fragments, derivatives, or analogs of the polypeptides of SEQ ID NO: 2, 4, 6, 8, 10, 12, 14, 16, 18, 20, or 22 can be substituted with one or more conserved or non-conserved amino acid residue (preferably a conserved amino acid residue). In some cases these polypeptides, fragments, derivatives, or analogs thereof will have a polypeptide sequence at least 50%, 55%, 60%, 65%, 70%, 75%, 80%, 85%, 90%, 91%, 92%, 93%, 94%, 95%, 96%, 97%, 98%, or 99% identical to the polypeptide sequence shown in SEQ ID NO: 2, 4, 6, 8, 10, 12, 14, 16, 18, 20, or 22 and will comprise functional or non-functional proteins or enzymes. Similarly, additions or deletions to the polypeptides can be made either at the N- or C-termini or within non-conserved regions of the polypeptide (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All the various embodiments, aspects, and options disclosed herein can be combined in any variations or iterations. The scope of the compositions, formulations, methods, and processes described herein include all actual or potential combinations of embodiments, aspects, options, examples, and preferences herein described. The compositions, formulations, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the specification discloses and describes merely exemplary embodiments. All patents and publications cited herein are incorporated by reference herein for the specific teachings thereof.

Various embodiments and aspects of the inventions described herein are summarized by the following clauses:
Clause 1. A nucleotide sequence encoding a polypeptide, where the polypeptide comprises one or more immunoglobulin domains comprising a fusion of the wild type N- and C-termini or an optional a linker joining the wild type N- and C-termini and a scission within one of the loop regions yielding novel N′- and C′-termini.
Clause 2. The nucleotide sequence of clause 1, wherein the immunoglobulin domain comprises an immunoglobulin domain from an immunoglobulin, Fab, Fv, T cell receptor (TCR), CD80, CTLA-4, PD1, PDL1, MHC molecules, or other immunoglobulin domain containing proteins.
Clause 3. The nucleotide sequence of clause 1 or 2, wherein the immunoglobulin domain comprises a heavy chain variable domain or a light chain variable domain.
Clause 4. The nucleotide sequence of any one of clauses 1-3, wherein one or both of the N′- and C′-termini are fused with one or more additional polypeptides.
Clause 5. The nucleotide sequence of any one of clauses 1-4, wherein the additional polypeptide comprises an immunoglobulin domain, Fab, Fv, ScFV, cell receptor, pMHC, costimulatory molecule, cytokine, or another polypeptide domain.
Clause 6. The nucleotide sequence of any one of clauses 1-4, wherein the additional polypeptide comprises an immunoglobulin hinge region.
Clause 7. The nucleotide sequence of any one of clauses 1-4, wherein the additional polypeptide comprises one or more of: CD80, CD86, ICAM-1, PD-L1/L2, B7H1, B7H2, CD40, CD40L, CD47, CD48, CD58, 4-1BBL, OX40L, TIM-1, TIM-4, CD80:PD-L1 heterodimer, calreticulin, a peptide that is at least 90% identical to CD80, CD86, ICAM-1, PD-L1/L2, B7H1, B7H2, CD40, CD40L, CD47, CD48, CD58, 4-1BBL, OX40L, TIM-1, TIM-4, CD80:PD-L1 heterodimer, calreticulin, fragments thereof, or combinations thereof; cytokines: IFNα, IFNβ, IFNγ, IL-1, IL-1a, IL-2, IL-4, IL-6, IL-7, IL-10, IL-12, IL-15, IL-21, IL-23, TNF, TNFα, TGFβ, GM-CSF, CSF-1, a peptide that is at least 90% identical to IFNα, IFNβ, IFNγ, IL-1, IL-1a, IL-2, IL-4, IL-6, IL-7, IL-10, IL-12, IL-15, IL-21, IL-23, TNF, TNFα, TGFβ, GM-CSF, CSF-1, fragments thereof, or combinations thereof; MHC alleles: MHC molecule comprises HLA-A, HLA-B, HLA-C, β2-microglobulin, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ, a peptide that is at least 90% identical to HLA-A, HLA-B, HLA-C, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ, fragments thereof, or combinations thereof; or TCR molecules: TRAC, TRBC1, TRBC2, TRDC, TRGC1, TRGC2, TCRA, TCB1, TCB2, TCC1, TCC2, TCC3, TCC4, a peptide that is at least 90% identical to TRAC, TRBC1, TRBC2, TRDC, TRGC1, TRGC2, TCRA, TCB1, TCB2, TCC1, TCC2, TCC3, TCC4, fragments thereof, or combinations thereof.
Clause 8. The nucleotide sequence of any one of clauses 1-7, wherein the linker comprises a polypeptide linker or a chemical linker.
Clause 9. The nucleotide sequence of any one of clauses 1-8, wherein the linker comprises a polypeptide selected from one or more of a poly glycine linker, poly alanine linker, poly glycine-alanine linker, poly glycine-serine linker, or poly glycine-serine-proline linker.
Clause 10. The nucleotide sequence of any one of clauses 1-9, wherein the linker comprises a polypeptide having 85% to 99% identity to one or more of SEQ ID NO: 36, 38, 40, 42, 44, 46, 48, 50, 52, 54, 56, or 58.
Clause 11. The nucleotide sequence of any one of clauses 1-10, wherein the linker is a polypeptide selected from one or more of SEQ ID NO: 36, 38, 40, 42, 44, 46, 48, 50, 52, 54, 56, or 58.
Clause 12. The nucleotide sequence of any one of clauses 1-11, wherein the loop region comprises one or more of the loops connecting adjacent β-strands comprising A-B, B-C, C-C′, C′-C″, C-D, C′-D, C″-D, D-E, E-F, F-G, or other loop-linkages that eliminate one or more intervening β-strands.
Clause 13. The nucleotide sequence of any one of clauses 1-12, wherein the loop region comprises one or more of the immunoglobulin β-sheet loops A-B, B-C, C-C′, C′-C″, C-D, C′-D, C″-D, D-E, E-F, or F-G.
Clause 14. The nucleotide sequence of any one of clauses 1-13, wherein the loop region comprises the C-C′ or A-B loop.
Clause 15. The nucleotide sequence of any one of clauses 1-14, wherein the nucleotide sequence has 85% to 99% identity to SEQ ID NO: 1, 3, 5, 7, 9, 11, 13, 15, 17, 19, 21, 23, 25, 27, 29, 31, or 33.
Clause 16. The nucleotide sequence of any one of clauses 1-15, wherein the nucleotide sequence is selected from SEQ ID NO: 1, 3, 5, 7, 9, 11, 13, 15, 17, 19, 21, 23, 25, 27, 29, 31, or 33.
Clause 17. A polynucleotide vector comprising one or more nucleotide sequences of any one of clauses 1-16.
Clause 18. A cell comprising one or more nucleotide sequences of clause 1 or a polynucleotide vector of clause 17.
Clause 19. A polypeptide encoded by the nucleotide sequence of any one of clauses 1-16.
Clause 20. A polypeptide encoded by the nucleotide sequence of any one of clauses 1-16, wherein the polypeptide has 85% to 99% identity to SEQ ID NO: 2, 4, 6, 8, 10, 12, 14, 16, 18, 20, 22, 24, 26, 28, 30, 32, or 34.
Clause 21. A polypeptide encoded by the nucleotide sequence of any one of clauses 1-16, wherein the polypeptide is selected from SEQ ID NO: 2, 4, 6, 8, 10, 12, 14, 16, 18, 20, 22, 24, 26, 28, 30, 32, or 34.
Clause 22. A bivalent polypeptide complex comprising a dimer of the polypeptides of SEQ ID NO: 32 or 34 covalently linked via one or more disulfide bonds.
Clause 23. A single chain variable fragment (scFv) polypeptide comprising SEQ ID NO: 34.
Clause 24. A process for manufacturing the nucleotide sequence of any one of clauses 1-16 or a polypeptide encoded by the nucleotide sequence of any one of clauses 1-16, the process comprising: transforming or transfecting a cell with the nucleic acid; growing the cells; optionally isolating additional quantities of the nucleotide sequence; inducing expression of the polypeptide; and isolating the polypeptide.
Clause 25. A means for manufacturing the nucleotide sequence of any one of clauses 1-16 or a polypeptide encoded by the nucleotide sequence of any one of clauses 1-16, the means comprising: transforming or transfecting a cell with the nucleic acid; growing the cells; optionally isolating additional quantities of the nucleotide sequence; inducing expression of the polypeptide; and isolating the polypeptide.
Clause 26. The nucleotide sequence of any one of clauses 1-16 or a polypeptide encoded by the nucleotide sequence of any one of clauses 1-16 produced by the process of clause 21 or the means of clause 22.
Clause 27. A method of treatment comprising administering an effective amount of polypeptide encoded by the nucleotide sequence of any one of clauses 1-16 to a subject in need thereof.
Clause 28. Use of an effective amount of a polypeptide encoded by the nucleotide sequence of any one of clauses 1-16 for the treatment of a disease or disorder comprising administering an effective amount the polypeptide to a subject in need thereof.
Clause 29. A research tool comprising a polypeptide encoded by the nucleotide sequence of any one of clauses 1-16.
Clause 30. An immunochemical reagent comprising a polypeptide encoded by the nucleotide sequence of any one of clauses 1-16.

### EXAMPLES

### Example 1

### AltIg and BMiMS Constructs

AltIg and BMiMS constructs were generated using standard molecular biology techniques. Genes (cDNA) encoding AltIg versions of the light chain (LC) and heavy chain (HC) Fv fragments of the anti-human CD19 monoclonal antibody B43 were purchased from IDT, cloned into pUC18 (Fermentas), sequenced (Eton Biosciences), and subcloned into an MSCV-based retroviral expression vector. For BMiMS, genes encoding the fusion partners for the AltIgs (e.g., mMHCII, mCD80, mCD86, and mICAM-1) were amplified by PCR with primers encoding the appropriate linkers to clone in-frame with the AltIg and encode the desired BMiMS. The PCR products (cDNA) were cloned into pUC18 (Fermentas), sequenced (Eton Biosciences), and then subcloned into an MSCV-based retroviral expression vector with the desired AltIg gene.

The constructs and their nucleotide (cDNA) and amino acid sequences are shown. The lowercase characters indicate inserted nucleotides or the linker regions in the polypeptide sequence.

mAbB43Fv-HC.Altlg SEQ ID NO: 1; 597 NT
ATGGTGTGGCTCCCCAGAGTTCCCTGTGTGGCAGCTGTGATCCTGTTGCTGACAGTGCTGAGCCCTCCAGTGGCTT
TGGTCAGAGACTCCTACCCATACGATGTTCCAGATTACGCTGGAGGTTCCGCGGCCGCAGGACAGGGTCTTGAGTG
GATTGGACAGATTTGGCCTGGAGATGGTGATACTAACTACAATGGAAAGTTCAAGGGTAAAGCCACTCTGACTGCA
GACGAATCCTCCAGCACAGCCTACATGCAACTCAGCAGCCTACGATCTGAGGACTCTGCGGTCTATTCTTGTGCAA
GACGGGAGACTACGACGGTAGGCCGTTATTACTATGCTATGGACTACTGGGGCCAAGGGACCACGGTCACCTCTCC
TGGATCCGGTGGAGGCGGATCAGGTGGCGGTGGAAGTGGAGGTGGTGGATCTTCCGGACAAGTGCAGCTCCTGGAG
TCTGGGGCTGAGCTGGTGAGGCCTGGGTCCTCAGTGAAGATTTCCTGCAAGGCTTCTGGCTATGCATTCAGTAGCT
ACTGGATGAACTGGGTGAAGCAGAGGGGTGGAGGCGGTTCCCATCACCATCACCATCACTGATGA
SEQ ID NO: 2; 197 AA
MVWLPRVPCVAAVILLLTVLSPPVALVRDSYPYDVPDYAGGSAAAGQGLEWIGQIWPGDGDTNYNGKFKGKATLTA
DESSSTAYMQLSSLRSEDSAVYSCARRETTTVGRYYYAMDYWGQGTTVTSPGSGGGGSGGGGSGGGGSSGQVQLLE
SGAELVRPGSSVKISCKASGYAFSSYWMNWVKQRGGGGSHHHHHH**
mAbB43Fv-LC.Altlg SEQ ID NO: 3; 570 NT
ATGGTGTGGCTCCCCAGAGTTCCCTGTGTGGCAGCTGTGATCCTGTTGCTGACAGTGCTGAGCCCTCCAGTGGCTT
TGGTCAGAGACTCCGAGCAGAAGCTGATCAGCGAGGAGGACCTGGGAGGTTCCGCGGCCGCAGGACAGCCACCCAA
ACTCCTCATCTATGATGCATCCAATCTAGTTTCTGGGATTCCACCCAGGTTTAGTGGCAGTGGGTCTGGGACAGAC
TTCACCCTCAACATCCATCCTGTGGAGAAGGTGGATGCTGCAACCTATCACTGTCAGCAAAGTACTGAAGATCCGT
GGACGTTCGGTGGAGGGACCAAGCTGGAAATAAAATCTCCTGGATCCGGTGGAGGCGGATCAGGTGGCGGTGGAAG
TGGAGGTGGTGGATCTTCCGGAGAGCTCGTGCTCACCCAGTCTCCAGCTTCTTTGGCTGTGTCTCTAGGGCAGAGG
GCCACCATCTCCTGCAAGGCCAGCCAAAGTGTTGATTATGATGGTGATAGTTATTTGAACTGGTACCAACAGATTG
GTGGAGGCGGTTCCCATCACCATCACCATCACTGATGA
SEQ ID NO: 4; 188 AA
MVWLPRVPCVAAVILLLTVLSPPVALVRDSEQKLISEEDLGGSAAAGQPPKLLIYDASNLVSGIPPRFSGSGSGTD
FTLNIHPVEKVDAATYHCQQSTEDPWTFGGGTKLEIKSPGSGGGGSGGGGSGGGGSSGELVLTQSPASLAVSLGQR
ATISCKASQSVDYDGDSYLNWYQQIGGGGSHHHHHH**
sCD80-hCD19LCFvFuse SEQ ID NO: 5; 1203 NT
ATGGCTTGCAATTGTCAGTTGATGCAGGATACACCACTCCTCAAGTTTCCATGTCCAAGGCTCATTCTTCTCTTTG
TGCTGCTGATTCGTCTTTCACAAGTGTCTTCAGATGTTGATGAACAACTGTCCAAGTCAGTGAAAGATAAGGTATT
GCTGCCTTGCCGTTACAACTCTCCTCATGAAGATGAGTCTGAAGACCGAATCTACTGGCAAAAACATGACAAAGTG
GTGCTGTCTGTCATTGCTGGGAAACTAAAAGTGTGGCCCGAGTATAAGAACCGGACTTTATATGACAACACTACCT
ACTCTCTTATCATCCTGGGCCTGGTCCTTTCAGACCGGGGCACATACAGCTGTGTCGTTCAAAAGAAGGAAAGAGG
AACGTATGAAGTTAAACACTTGGCTTTAGTAAAGTTGTCCATCAAAGCTGACTTCTCTACCCCCAACATAACTGAG
TCTGGAAACCCATCTGCAGACACTAAAAGGATTACCTGCTTTGCTTCCGGGGGTTTCCCAAAGCCTCGCTTCTCTT
GGTTGGAAAATGGAAGAGAATTACCTGGCATCAATACGACAATTTCCCAGGATCCTGAATCTGAATTGTACACCAT
TAGTAGCCAACTAGATTTCAATACGACTCGCAACCACACCATTAAGTGTCTCATTAAATATGGAGATGCTCACGTG
TCAGAGGACTTCACCTGGGAAAAACCCCCAGAAGACCCTCCTGATAGCAAGAACACAgcggccgcaGGACAGCCAC
CCAAACTCCTCATCTATGATGCATCCAATCTAGTTTCTGGGATTCCACCCAGGTTTAGTGGCAGTGGGTCTGGGAC
AGACTTCACCCTCAACATCCATCCTGTGGAGAAGGTGGATGCTGCAACCTATCACTGTCAGCAAAGTACTGAAGAT
CCGTGGACGTTCGGTGGAGGGACCAAGCTGGAAATAAAATCTcctggatccggtggaggcggatcaggtggcggtg
gaagtggaggtggtggatcttccggaGAGCTCGTGCTCACCCAGTCTCCAGCTTCTTTGGCTGTGTCTCTAGGGCA
GAGGGCCACCATCTCCTGCAAGGCCAGCCAAAGTGTTGATTATGATGGTGATAGTTATTTGAACTGGTACCAACAG
ATTggtggaggcggttccCATCACCATCACCATCACtgatgaagatctcaattggaattctga
SEQ ID NO: 6; 392 AA
MACNCQLMQDTPLLKFPCPRLILLFVLLIRLSQVSSDVDEQLSKSVKDKVLLPCRYNSPHEDESEDRIYWQKHDKV
VLSVIAGKLKVWPEYKNRTLYDNTTYSLIILGLVLSDRGTYSCVVQKKERGTYEVKHLALVKLSIKADFSTPNITE
SGNPSADTKRITCFASGGFPKPRFSWLENGRELPGINTTISQDPESELYTISSQLDFNTTRNHTIKCLIKYGDAHV
SEDFTWEKPPEDPPDSKNTAAAGQPPKLLIYDASNLVSGIPPRFSGSGSGTDFTLNIHPVEKVDAATYHCQQSTED
PWTFGGGTKLEIKSPGSGGGGSGGGGSGGGGSSGELVLTQSPASLAVSLGQRATISCKASQSVDYDGDSYLNWYQQ
IGGGGSHHHHHH**
sCD80-hCD19HCFvFuse SEQ ID NO: 7; 1233 NT
ATGGCTTGCAATTGTCAGTTGATGCAGGATACACCACTCCTCAAGTTTCCATGTCCAAGGATGGCTTGCAATTGTC
AGTTGATGCAGGATACACCACTCCTCAAGTTTCCATGTCCAAGGCTCATTCTTCTCTTTGTGCTGCTGATTCGTCT
TTCACAAGTGTCTTCAGATGTTGATGAACAACTGTCCAAGTCAGTGAAAGATAAGGTATTGCTGCCTTGCCGTTAC
AACTCTCCTCATGAAGATGAGTCTGAAGACCGAATCTACTGGCAAAAACATGACAAAGTGGTGCTGTCTGTCATTG
CTGGGAAACTAAAAGTGTGGCCCGAGTATAAGAACCGGACTTTATATGACAACACTACCTACTCTCTTATCATCCT
GGGCCTGGTCCTTTCAGACCGGGGCACATACAGCTGTGTCGTTCAAAAGAAGGAAAGAGGAACGTATGAAGTTAAA
CACTTGGCTTTAGTAAAGTTGTCCATCAAAGCTGACTTCTCTACCCCCAACATAACTGAGTCTGGAAACCCATCTG
CAGACACTAAAAGGATTACCTGCTTTGCTTCCGGGGGTTTCCCAAAGCCTCGCTTCTCTTGGTTGGAAAATGGAAG
AGAATTACCTGGCATCAATACGACAATTTCCCAGGATCCTGAATCTGAATTGTACACCATTAGTAGCCAACTAGAT
TTCAATACGACTCGCAACCACACCATTAAGTGTCTCATTAAATATGGAGATGCTCACGTGTCAGAGGACTTCACCT
GGGAAAAACCCCCAGAAGACCCTCCTGATAGCAAGAACACAgcggccgcaGGACAGGGTCTTGAGTGGATTGGACA
GATTTGGCCTGGAGATGGTGATACTAACTACAATGGAAAGTTCAAGGGTAAAGCCACTCTGACTGCAGACGAATCC
TCCAGCACAGCCTACATGCAACTCAGCAGCCTACGATCTGAGGACTCTGCGGTCTATTCTTGTGCAAGACGGGAGA
CTACGACGGTAGGCCGTTATTACTATGCTATGGACTACTGGGGCCAAGGGACCACGGTCACCTCTcctggatccgg
tggaggcggatcaggtggcggtggaagtggaggtggtggatcttccggaCAAGTGCAGCTCCTGGAGTCTGGGGCT
GAGCTGGTGAGGCCTGGGTCCTCAGTGAAGATTTCCTGCAAGGCTTCTGGCTATGCATTCAGTAGCTACTGGATGA
ACTGGGTGAAGCAGAGGggtggaggcggttccCATCACCATCACCATCACtgatgaagatctcaattggaattctg
a
SEQ ID NO: 8; 402 AA
MACNCQLMQDTPLLKFPCPRLILLFVLLIRLSQVSSDVDEQLSKSVKDKVLLPCRYNSPHEDESEDRIYWQKHDKV
VLSVIAGKLKVWPEYKNRTLYDNTTYSLIILGLVLSDRGTYSCVVQKKERGTYEVKHLALVKLSIKADFSTPNITE
SGNPSADTKRITCFASGGFPKPRFSWLENGRELPGINTTISQDPESELYTISSQLDFNTTRNHTIKCLIKYGDAHV
SEDFTWEKPPEDPPDSKNTAAAGQGLEWIGQIWPGDGDTNYNGKFKGKATLTADESSSTAYMQLSSLRSEDSAVYS
CARRETTTVGRYYYAMDYWGQGTTVTSPGSGGGGSGGGGSGGGGSSGQVQLLESGAELVRPGSSVKISCKASGYAF
SSYWMNWVKQRGGGGSHHHHHH**
sCD86-hCD19HCFvFuse SEQ ID NO: 9; 1227 NT
ATGGACCCCAGATGCACcatgggcttggcaatccttatctttgtgacagtcttgctgatctcagatgctgtttccg
tggagacgcaagcttatttcaatgggactgcatatctgccgtgcccatttacaaaggctcaaaacataagcctgag
tgagctggtagtattttggcaggaccagcaaaagttggttctgtacgagcactatttgggcacagagaaacttgat
agtgtgaatgccaagtacctgggccgcacgagctttgacaggaacaactggactctacgacttcacaatgttcaga
tcaaggacatgggctcgtatgattgttttatacaaaaaaagccacccacaggatcaattatcctccaacagacatt
aacagaactgtcagtgatcgccaacttcagtgaacctgaaataaaactggctcagaatgtaacaggaaattctggc
ataaatttgacctgcacgtctaagcaaggtcacccgaaacctaagaagatgtattttctgataactaattcaacta
atgagtatggtgataacatgcagatatcacaagataatgtcacagaactgttcagtatctccaacagcctctctct
ttcattcccggatggtgtgtggcatatgaccgttgtgtgtgttctggaaacggagtcaatgaagatttcctccaaa
cctctcaatttcactcaagagtttccatctcCTCAAACGTATTGGAAGGAGgcggccgcaGGACAGGGTCTTGAGT
GGATTGGACAGATTTGGCCTGGAGATGGTGATACTAACTACAATGGAAAGTTCAAGGGTAAAGCCACTCTGACTGC
AGACGAATCCTCCAGCACAGCCTACATGCAACTCAGCAGCCTACGATCTGAGGACTCTGCGGTCTATTCTTGTGCA
AGACGGGAGACTACGACGGTAGGCCGTTATTACTATGCTATGGACTACTGGGGCCAAGGGACCACGGTCACCTCTc
ctggatccggtggaggcggatcaggtggcggtggaagtggaggtggtggatcttccggaCAAGTGCAGCTCCTGGA
GTCTGGGGCTGAGCTGGTGAGGCCTGGGTCCTCAGTGAAGATTTCCTGCAAGGCTTCTGGCTATGCATTCAGTAGC
TACTGGATGAACTGGGTGAAGCAGAGGggtggaggcggttccCATCACCATCACCATCACtgatgaagatctcaat
tggaattctga
SEQ ID NO: 10; 400 AA
MDPRCTMGLAILIFVTVLLISDAVSVETQAYFNGTAYLPCPFTKAQNISLSELVVFWQDQQKLVLYEHYLGTEKLD
SVNAKYLGRTSFDRNNWTLRLHNVQIKDMGSYDCFIQKKPPTGSIILQQTLTELSVIANFSEPEIKLAQNVTGNSG
INLTCTSKQGHPKPKKMYFLITNSTNEYGDNMQISQDNVTELFSISNSLSLSFPDGVWHMTVVCVLETESMKISSK
PLNFTQEFPSPQTYWKEAAAGQGLEWIGQIWPGDGDTNYNGKFKGKATLTADESSSTAYMQLSSLRSEDSAVYSCA
RRETTTVGRYYYAMDYWGQGTTVTSPGSGGGGSGGGGSGGGGSSGQVQLLESGAELVRPGSSVKISCKASGYAFSS
YWMNWVKQRGGGGSHHHHHH**
SICAM1-hCD19HCFvFuse SEQ ID NO: 11; 1953 NT
ATGGCTTCAACCCGTGCCAAGCCCACGCTACCTCTGCTCCTGGCCCTGGTCACCGTTGTGATCCCTGGGCCTGGTG
ATGCTCAGGTATCCATCCATCCCAGAGAAGCCTTCCTGCCCCAGGGTGGGTCCGTGCAGGTGAACTGTTCTTCCTC
ATGCAAGGAGGACCTCAGCCTGGGCTTGGAGACTCAGTGGCTGAAAGATGAGCTCGAGAGTGGACCCAACTGGAAG
CTGTTTGAGCTGAGCGAGATCGGGGAGGACAGCAGTCCGCTGTGCTTTGAGAACTGTGGCACCGTGCAGTCGTCCG
CTTCCGCTACCATCACCGTGTATTCGTTTCCGGAGAGTGTGGAGCTGAGACCTCTGCCAGCCTGGCAGCAAGTAGG
CAAGGACCTCACCCTGCGCTGCCACGTGGATGGTGGAGCACCGCGGACCCAGCTCTCAGCAGTGCTGCTCCGTGGG
GAGGAGATACTGAGCCGCCAGCCAGTGGGTGGGCACCCCAAGGACCCCAAGGAGATCACATTCACGGTGCTGGCTA
GCAGAGGGGACCACGGAGCCAATTTCTCATGCCGCACAGAACTGGATCTCAGGCCGCAAGGGCTGGCATTGTTCTC
TAATGTCTCCGAGGCCAGGAGCCTCCGGACTTTCGATCTTCCAGCTACCATCCCAAAGCTCGACACCCCTGACCTC
CTGGAGGTGGGCACCCAGCAGAAGTTGTTTTGCTCCCTGGAAGGCCTGTTTCCTGCCTCTGAAGCTCGGATATACC
TGGAGCTGGGAGGCCAGATGCCGACCCAGGAGAGCACAAACAGCAGTGACTCTGTGTCAGCCACTGCCTTGGTAGA
GGTGACTGAGGAGTTCGACAGAACCCTGCCGCTGCGCTGCGTTTTGGAGCTAGCGGACCAGATCCTGGAGACGCAG
AGGACCTTAACAGTCTACAACTTTTCAGCTCCGGTCCTGACCCTGAGCCAGCTGGAGGTCTCGGAAGGGAGCCAAG
TAACTGTGAAGTGTGAAGCCCACAGTGGGTCGAAGGTGGTTCTTCTGAGCGGCGTCGAGCCTAGGCCACCCACCCC
GCAGGTCCAATTCACACTGAATGCCAGCTCGGAGGATCACAAACGAAGCTTCTTTTGCTCTGCCGCTCTGGAGGTG
GCGGGAAAGTTCCTGTTTAAAAACCAGACCCTGGAACTGCACGTGCTGTATGGTCCTCGGCTGGACGAGACGGACT
GCTTGGGGAACTGGACCTGGCAAGAGGGGTCTCAGCAGACTCTGAAATGCCAGGCCTGGGGGAACCCATCTCCTAA
GATGACCTGCAGACGGAAGGCAGATGGTGCCCTGCTGCCCATCGGGGTGGTGAAGTCTGTCAAACAGGAGATGAAT
GGTACATACGTGTGCCATGCCTTTAGCTCCCATGGGAATGTCACCAGGAATGTGTACCTGACAGTACTGTACCACT
CTCAAAATAACTGGACTgcggccgcaGGACAGGGTCTTGAGTGGATTGGACAGATTTGGCCTGGAGATGGTGATAC
TAACTACAATGGAAAGTTCAAGGGTAAAGCCACTCTGACTGCAGACGAATCCTCCAGCACAGCCTACATGCAACTC
AGCAGCCTACGATCTGAGGACTCTGCGGTCTATTCTTGTGCAAGACGGGAGACTACGACGGTAGGCCGTTATTACT
ATGCTATGGACTACTGGGGCCAAGGGACCACGGTCACCTCTcctggatccggtggaggcggatcaggtggcggtgg
aagtggaggtggtggatcttccggaCAAGTGCAGCTCCTGGAGTCTGGGGCTGAGCTGGTGAGGCCTGGGTCCTCA
GTGAAGATTTCCTGCAAGGCTTCTGGCTATGCATTCAGTAGCTACTGGATGAACTGGGTGAAGCAGAGGggtggag
gcggttccCATCACCATCACCATCACtgatgaagatctcaattggaattctga
SEQ ID NO: 12; 642 AA
MASTRAKPTLPLLLALVTVVIPGPGDAQVSIHPREAFLPQGGSVQVNCSSSCKEDLSLGLETQWLKDELESGPNWK
LFELSEIGEDSSPLCFENCGTVQSSASATITVYSFPESVELRPLPAWQQVGKDLTLRCHVDGGAPRTQLSAVLLRG
EEILSRQPVGGHPKDPKEITFTVLASRGDHGANFSCRTELDLRPQGLALFSNVSEARSLRTFDLPATIPKLDTPDL
LEVGTQQKLFCSLEGLFPASEARIYLELGGQMPTQESTNSSDSVSATALVEVTEEFDRTLPLRCVLELADQILETQ
RTLTVYNFSAPVLTLSQLEVSEGSQVTVKCEAHSGSKVVLLSGVEPRPPTPQVQFTLNASSEDHKRSFFCSAALEV
AGKFLFKNQTLELHVLYGPRLDETDCLGNWTWQEGSQQTLKCQAWGNPSPKMTCRRKADGALLPIGVVKSVKQEMN
GTYVCHAFSSHGNVTRNVYLTVLYHSQNNWTAAAGQGLEWIGQIWPGDGDTNYNGKFKGKATLTADESSSTAYMQL
SSLRSEDSAVYSCARRETTTVGRYYYAMDYWGQGTTVTSPGSGGGGSGGGGSGGGGSSGQVQLLESGAELVRPGSS
VKISCKASGYAFSSYWMNWVKQRGGGGSHHHHHH**
SIE^{k}α (MHCIIα) acid zipper-hCD19LCFvFuse SEQ ID NO: 13; 1254 NT
atggctacaaggctcctctgttacacagtactttgtctcctgggtgcaagaattttgaattgtATCAAAGAGGAAC
ACACCATCatccaggcagagttctatcttttaccagacaaacgtggagagtttatgtttgactttgacggcgatga
gattttccatgtagacattgaaaagtcagagaccatctggagacttgaagaatttgcaaagtttgccagctttgag
gctcagggtgcactggctaatatagctgtggacaaagctaacctggatgtcatgaaagagcgttccaacaacactc
cagatgccaacgtggccccagaggtgactgtactctccagaagccctgtgaacctgggagagcccaacatcctcat
ctgtttcattgacaagttctcccctccagtggtcaatgtcacctggctccggaatggacggcctgtcaccgaaggc
gtgtcagagacagtgtttctcccgagggacgatcacctcttccgcaaattccactatctgaccttcctgccctcca
cagatgatttctatgactgtgaggtggatcactggggcttggaggagcctctgcggaagcactgggagtttgaaga
gaaaaccctcctcccagaaactaaagagtctagaggtggcctggaagttctgttccaggggcccgaattcggcggt
tccactacagctccatcagctcagctcgaaaaagagctccaggccctggagaaggaaaatgcacagctggaatggg
agttgcaagcactGGAAAAGGAACTGGCTCAGgcggccgcaGGACAGCCACCCAAACTCCTCATCTATGATGCATC
CAATCTAGTTTCTGGGATTCCACCCAGGTTTAGTGGCAGTGGGTCTGGGACAGACTTCACCCTCAACATCCATCCT
GTGGAGAAGGTGGATGCTGCAACCTATCACTGTCAGCAAAGTACTGAAGATCCGTGGACGTTCGGTGGAGGGACCA
AGCTGGAAATAAAATCTcctggatccggtggaggcggatcaggtggcggtggaagtggaggtggtggatcttccgg
aGAGCTCGTGCTCACCCAGTCTCCAGCTTCTTTGGCTGTGTCTCTAGGGCAGAGGGCCACCATCTCCTGCAAGGCC
AGCCAAAGTGTTGATTATGATGGTGATAGTTATTTGAACTGGTACCAACAGATTggtggaggcggttccCATCACC
ATCACCATCACtgatgaagatctcaattggaattctga
SEQ ID NO: 14; 409 AA
MATRLLCYTVLCLLGARILNCIKEEHTIIQAEFYLLPDKRGEFMFDFDGDEIFHVDIEKSETIWRLEEFAKFASFE
AQGALANIAVDKANLDVMKERSNNTPDANVAPEVTVLSRSPVNLGEPNILICFIDKFSPPVVNVTWLRNGRPVTEG
VSETVFLPRDDHLFRKFHYLTFLPSTDDFYDCEVDHWGLEEPLRKHWEFEEKTLLPETKESRGGLEVLFQGPEFGG
STTAPSAQLEKELQALEKENAQLEWELQALEKELAQAAAGQPPKLLIYDASNLVSGIPPRFSGSGSGTDFTLNIHP
VEKVDAATYHCQQSTEDPWTFGGGTKLEIKSPGSGGGGSGGGGSGGGGSSGELVLTQSPASLAVSLGQRATISCKA
SQSVDYDGDSYLNWYQQIGGGGSHHHHHH*
SIE^{k}β (MHCIIβ) basic zipper-hCD19HCFvFuse SEQ ID NO: 15; 1453 NT
atggctacaaggctcctctgttacacagtactttgtctcctgggtgcaagaattttgaattgtgcGGATCCCTCCG
GCTCCgccaacgagagggccgacctgatcgcctacctgaagcaggccaccaaggaattcagatccggaggcggagg
tgtcatttctacaacgggacgcagcgcgtgcggcttctggtaagatacttctacaacctggaggagaacctgcgct
tcgacagcgacgtgggcgagttccgcgcggtgaccgagctggggcggccagacgccgagaactggaacagccagcc
ggagttcctggagcaaaagcgggccgaggtggacacggtgtgcagacacaactatgagatcttcgataacttcctt
gtgccgcggagagttgagcctacggtgactgtgtaccccacaaagacgcagcccctggaacaccacaacctcctgg
tctgctctgtgagtgacttctaccctggcaacattgaagtcagatggttccggaatggcaaggaggagaaaacagg
aattgtgtccacgggcctggtccgaaatggagactggaccttccagacactggtgatgctggagacggttcctcag
agtggagaggtttacacctgccaggtggagcatcccagcctgaccgaccctgtcacggtcgagtggaaagcacagt
ccacatctgcacagaacaagtctagaggtggcctggaagttctgttccaggggcccgaattcggcggttccactac
agctccatcagctcagttgaaaaagaaattgcaagcactgaagaaaaagaacgctcagctgaagtggaaacttcaa
gccctCAAGAAGAAACTCGCCCAGgcggccgcaGGACAGGGTCTTGAGTGGATTGGACAGATTTGGCCTGGAGATG
GTGATACTAACTACAATGGAAAGTTCAAGGGTAAAGCCACTCTGACTGCAGACGAATCCTCCAGCACAGCCTACAT
GCAACTCAGCAGCCTACGATCTGAGGACTCTGCGGTCTATTCTTGTGCAAGACGGGAGACTACGACGGTAGGCCGT
TATTACTATGCTATGGACTACTGGGGCCAAGGGACCACGGTCACCTCTcctggatccggtggaggcggatcaggtg
gcggtggaagtggaggtggtggatcttccggaCAAGTGCAGCTCCTGGAGTCTGGGGCTGAGCTGGTGAGGCCTGG
GTCCTCAGTGAAGATTTCCTGCAAGGCTTCTGGCTATGCATTCAGTAGCTACTGGATGAACTGGGTGAAGCAGAGG
ggtggaggcggttccCATCACCATCACCATCACtgatgaagatctcaattggaattctga
SEQ ID NO: 16; 467 AA
MATRLLCYTVLCLLGARILNCADPSGSANERADLIAYLKQATKEFRSGGGGSLVPRGSGGGGSVDRPWFLEYCKSE
CHFYNGTQRVRLLVRYFYNLEENLRFDSDVGEFRAVTELGRPDAENWNSQPEFLEQKRAEVDTVCRHNYEIFDNFL
VPRRVEPTVTVYPTKTQPLEHHNLLVCSVSDFYPGNIEVRWFRNGKEEKTGIVSTGLVRNGDWTFQTLVMLETVPQ
SGEVYTCQVEHPSLTDPVTVEWKAQSTSAQNKSRGGLEVLFQGPEFGGSTTAPSAQLKKKLQALKKKNAQLKWKLQ
ALKKKLAQAAAGQGLEWIGQIWPGDGDTNYNGKFKGKATLTADESSSTAYMQLSSLRSEDSAVYSCARRETTTVGR
YYYAMDYWGQGTTVTSPGSGGGGSGGGGSGGGGSSGQVQLLESGAELVRPGSSVKISCKASGYAFSSYWMNWVKQR
GGGGSHHHHHH**
mAbB43FV-ABLoop Open hCD19HC Altlg SEQ ID NO: 17; 618 NT
ATGGTGTGGCTCCCCAGAGTTCCCTGTGTGGCAGCTGTGATCCTGTTGCTGACAGTGCTGAGCCCTCCAGTGGCTT
TGGTCAGAGACTCCTACCCATACGATGTTCCAGATTACGCTGGAGGTTCCGCGGCCGCAGGGTCCTCAGTGAAGAT
TTCCTGCAAGGCTTCTGGCTATGCATTCAGTAGCTACTGGATGAACTGGGTGAAGCAGAGGCCTGGACAGGGTCTT
GAGTGGATTGGACAGATTTGGCCTGGAGATGGTGATACTAACTACAATGGAAAGTTCAAGGGTAAAGCCACTCTGA
CTGCAGACGAATCCTCCAGCACAGCCTACATGCAACTCAGCAGCCTACGATCTGAGGACTCTGCGGTCTATTCTTG
TGCAAGACGGGAGACTACGACGGTAGGCCGTTATTACTATGCTATGGACTACTGGGGCCAAGGGACCACGGTCACC
TCTCCTGGATCCGGTGGAGGCGGATCAGGTGGCGGTGGAAGTGGAGGTGGTGGATCTTCCGGACAAGTGCAGCTCC
TGGAGTCTGGGGCTGAGCTGGTGAGGGGTGGAGGCGGTTCCCATCACCATCACCATCACTGATGAAGATCTCAATT
GGAATTCTGA
SEQ ID NO: 18; 197 AA
MVWLPRVPCVAAVILLLTVLSPPVALVRDSYPYDVPDYAGGSAAAGSSVKISCKASGYAFSSYWMNWVKQRPGQGL
EWIGQIWPGDGDTNYNGKFKGKATLTADESSSTAYMQLSSLRSEDSAVYSCARRETTTVGRYYYAMDYWGQGTTVT
SPGSGGGGSGGGGSGGGGSSGQVQLLESGAELVRGGGGSHHHHHH**
mAbB43FV-ABLoop Open hCD19LC Altlg SEQ ID NO: 19; 594 NT
ATGGTGTGGCTCCCCAGAGTTCCCTGTGTGGCAGCTGTGATCCTGTTGCTGACAGTGCTGAGCCCTCCAGTGGCTT
TGGTCAGAGACTCCGAGCAGAAGCTGATCAGCGAGGAGGACCTGGGAGGTTCCGCGGCCGCAGGGCAGAGGGCCAC
CATCTCCTGCAAGGCCAGCCAAAGTGTTGATTATGATGGTGATAGTTATTTGAACTGGTACCAACAGATTCCAGGA
CAGCCACCCAAACTCCTCATCTATGATGCATCCAATCTAGTTTCTGGGATCCCACCCAGGTTTAGTGGCAGTGGGT
CTGGGACAGACTTCACCCTCAACATCCATCCTGTGGAGAAGGTGGATGCTGCAACCTATCACTGTCAGCAAAGTAC
TGAGGATCCGTGGACGTTCGGTGGAGGGACCAAGCTGGAAATAAAATCTCCTGGATCCGGTGGAGGCGGATCAGGT
GGCGGTGGAAGTGGAGGTGGTGGATCTTCCGGAGAGCTCGTGCTCACCCAGTCTCCAGCTTCTTTGGCTGTGTCTC
TAGGTGGAGGCGGTTCCCATCACCATCACCATCACTGATGAAGATCTCAATTGGAATTCTGA
SEQ ID NO: 20; 190 AA
MVWLPRVPCVAAVILLLTVLSPPVALVRDSEQKLISEEDLGGSAAAGQRATISCKASQSVDYDGDSYLNWYQQIPG
QPPKLLIYDASNLVSGIPPRFSGSGSGTDFTLNIHPVEKVDAATYHCQQSTEDPWTFGGGTKLEIKSPGSGGGGSG
GGGSGGGGSSGELVLTQSPASLAVSLGGGGSHHHHHH**
sCD80-ABLoop Open hCD19HC Altlg SEQ ID NO: 21; 1233 NT
ATGGCTTGCAATTGTCAGTTGATGCAGGATACACCACTCCTCAAGTTTCCATGTCCAAGGCTCATTCTTCTCTTTG
TGCTGCTGATTCGTCTTTCACAAGTGTCTTCAGATGTTGATGAACAACTGTCCAAGTCAGTGAAAGATAAGGTATT
GCTGCCTTGCCGTTACAACTCTCCTCATGAAGATGAGTCTGAAGACCGAATCTACTGGCAAAAACATGACAAAGTG
GTGCTGTCTGTCATTGCTGGGAAACTAAAAGTGTGGCCCGAGTATAAGAACCGGACTTTATATGACAACACTACCT
ACTCTCTTATCATCCTGGGCCTGGTCCTTTCAGACCGGGGCACATACAGCTGTGTCGTTCAAAAGAAGGAAAGAGG
AACGTATGAAGTTAAACACTTGGCTTTAGTAAAGTTGTCCATCAAAGCTGACTTCTCTACCCCCAACATAACTGAG
TCTGGAAACCCATCTGCAGACACTAAAAGGATTACCTGCTTTGCTTCCGGGGGTTTCCCAAAGCCTCGCTTCTCTT
GGTTGGAAAATGGAAGAGAATTACCTGGCATCAATACGACAATTTCCCAGGATCCTGAATCTGAATTGTACACCAT
TAGTAGCCAACTAGATTTCAATACGACTCGCAACCACACCATTAAGTGTCTCATTAAATATGGAGATGCTCACGTG
TCAGAGGACTTCACCTGGGAAAAACCCCCAGAAGACCCTCCTGATAGCAAGAACACAgcggccgcagggtcctcag
tgaagatttcctgcaaggcttctggctatgcattcagtagctactggatgaactgggtgaagcagaggcctggaca
gggtcttgagtggattggacagatttggcctggagatggtgatactaactacaatggaaagttcaagggtaaagcc
actctgactgcagacgaatcctccagcacagcctacatgcaactcagcagcctacgatctgaggactctgcggtct
attcttgtgcaagacgggagactacgacggtaggccgttattactatgctatggactactggggccaagggaccac
ggtcacctctcctggatccggtggaggcggatcaggtggcggtggaagtggaggtggtggatcttccggacaagtg
cagctcctggagtctggggctgagctggtgaggggtggaggcggttcccatcaccatcaccatcactgatgaagat
ctcaattggaattctga
SEQ ID NO: 22; 403 AA
MACNCQLMQDTPLLKFPCPRLILLFVLLIRLSQVSSDVDEQLSKSVKDKVLLPCRYNSPHEDESEDRIYWQKHDKV
VLSVIAGKLKVWPEYKNRTLYDNTTYSLIILGLVLSDRGTYSCVVQKKERGTYEVKHLALVKLSIKADFSTPNITE
SGNPSADTKRITCFASGGFPKPRFSWLENGRELPGINTTISQDPESELYTISSQLDFNTTRNHTIKCLIKYGDAHV
SEDFTWEKPPEDPPDSKNTAAAGSSVKISCKASGYAFSSYWMNWVKQRPGQGLEWIGQIWPGDGDTNYNGKFKGKA
TLTADESSSTAYMQLSSLRSEDSAVYSCARRETTTVGRYYYAMDYWGQGTTVTSPGSGGGGSGGGGSGGGGSSGQV
QLLESGAELVRGGGGSHHHHHH**
sCD80-ABLoop Open hCD19LC Altlg SEQ ID NO: 23; 1206 NT
ATGGCTTGCAATTGTCAGTTGATGCAGGATACACCACTCCTCAAGTTTCCATGTCCAAGGCTCATTCTTCTCTTTG
TGCTGCTGATTCGTCTTTCACAAGTGTCTTCAGATGTTGATGAACAACTGTCCAAGTCAGTGAAAGATAAGGTATT
GCTGCCTTGCCGTTACAACTCTCCTCATGAAGATGAGTCTGAAGACCGAATCTACTGGCAAAAACATGACAAAGTG
GTGCTGTCTGTCATTGCTGGGAAACTAAAAGTGTGGCCCGAGTATAAGAACCGGACTTTATATGACAACACTACCT
ACTCTCTTATCATCCTGGGCCTGGTCCTTTCAGACCGGGGCACATACAGCTGTGTCGTTCAAAAGAAGGAAAGAGG
AACGTATGAAGTTAAACACTTGGCTTTAGTAAAGTTGTCCATCAAAGCTGACTTCTCTACCCCCAACATAACTGAG
TCTGGAAACCCATCTGCAGACACTAAAAGGATTACCTGCTTTGCTTCCGGGGGTTTCCCAAAGCCTCGCTTCTCTT
GGTTGGAAAATGGAAGAGAATTACCTGGCATCAATACGACAATTTCCCAGGATCCTGAATCTGAATTGTACACCAT
TAGTAGCCAACTAGATTTCAATACGACTCGCAACCACACCATTAAGTGTCTCATTAAATATGGAGATGCTCACGTG
TCAGAGGACTTCACCTGGGAAAAACCCCCAGAAGACCCTCCTGATAGCAAGAACACAgcggccgcAGGGCAGAGGG
CCACCATCTCCTGCAAGGCCAGCCAAAGTGTTGATTATGATGGTGATAGTTATTTGAACTGGTACCAACAGATTCC
AGGACAGCCACCCAAACTCCTCATCTATGATGCATCCAATCTAGTTTCTGGGATCCCACCCAGGTTTAGTGGCAGT
GGGTCTGGGACAGACTTCACCCTCAACATCCATCCTGTGGAGAAGGTGGATGCTGCAACCTATCACTGTCAGCAAA
GTACTGAGGATCCGTGGACGTTCGGTGGAGGGACCAAGCTGGAAATAAAATCTCCTGGATCCGGTGGAGGCGGATC
AGGTGGCGGTGGAAGTGGAGGTGGTGGATCTTCCGGAGAGCTCGTGCTCACCCAGTCTCCAGCTTCTTTGGCTGTG
TCTCTAGGTGGAGGCGGTTCCCATCACCATCACCATCACTGATGAAGATCTCAATTGGAATTCTGA
SEQ ID NO: 24; 394 AA
MACNCQLMQDTPLLKFPCPRLILLFVLLIRLSQVSSDVDEQLSKSVKDKVLLPCRYNSPHEDESEDRIYWQKHDKV
VLSVIAGKLKVWPEYKNRTLYDNTTYSLIILGLVLSDRGTYSCVVQKKERGTYEVKHLALVKLSIKADFSTPNITE
SGNPSADTKRITCFASGGFPKPRFSWLENGRELPGINTTISQDPESELYTISSQLDFNTTRNHTIKCLIKYGDAHV
SEDFTWEKPPEDPPDSKNTAAAGQRATISCKASQSVDYDGDSYLNWYQQIPGQPPKLLIYDASNLVSGIPPRFSGS
GSGTDFTLNIHPVEKVDAATYHCQQSTEDPWTFGGGTKLEIKSPGSGGGGSGGGGSGGGGSSGELVLTQSPASLAV
SLGGGGSHHHHHH**
sCD86-ABLoop Open hCD19HC Altlg SEQ ID NO: 25; 1227 NT
ATGGACCCCAGATGCACCATGGGCTTGGCAATCCTTATCTTTGTGACAGTCTTGCTGATCTCAGATGCTGTTTCCG
TGGAGACGCAAGCTTATTTCAATGGGACTGCATATCTGCCGTGCCCATTTACAAAGGCTCAAAACATAAGCCTGAG
TGAGCTGGTAGTATTTTGGCAGGACCAGCAAAAGTTGGTTCTGTACGAGCACTATTTGGGCACAGAGAAACTTGAT
AGTGTGAATGCCAAGTACCTGGGCCGCACGAGCTTTGACAGGAACAACTGGACTCTACGACTTCACAATGTTCAGA
TCAAGGACATGGGCTCGTATGATTGTTTTATACAAAAAAAGCCACCCACAGGATCAATTATCCTCCAACAGACATT
AACAGAACTGTCAGTGATCGCCAACTTCAGTGAACCTGAAATAAAACTGGCTCAGAATGTAACAGGAAATTCTGGC
ATAAATTTGACCTGCACGTCTAAGCAAGGTCACCCGAAACCTAAGAAGATGTATTTTCTGATAACTAATTCAACTA
ATGAGTATGGTGATAACATGCAGATATCACAAGATAATGTCACAGAACTGTTCAGTATCTCCAACAGCCTCTCTCT
TTCATTCCCGGATGGTGTGTGGCATATGACCGTTGTGTGTGTTCTGGAAACGGAGTCAATGAAGATTTCCTCCAAA
CCTCTCAATTTCACTCAAGAGTTTCCATCTCCTCAAACGTATTGGAAGGAGGCGGCCGCAGGGTCCTCAGTGAAGA
TTTCCTGCAAGGCTTCTGGCTATGCATTCAGTAGCTACTGGATGAACTGGGTGAAGCAGAGGCCTGGACAGGGTCT
TGAGTGGATTGGACAGATTTGGCCTGGAGATGGTGATACTAACTACAATGGAAAGTTCAAGGGTAAAGCCACTCTG
ACTGCAGACGAATCCTCCAGCACAGCCTACATGCAACTCAGCAGCCTACGATCTGAGGACTCTGCGGTCTATTCTT
GTGCAAGACGGGAGACTACGACGGTAGGCCGTTATTACTATGCTATGGACTACTGGGGCCAAGGGACCACGGTCAC
CTCTCCTGGATCCGGTGGAGGCGGATCAGGTGGCGGTGGAAGTGGAGGTGGTGGATCTTCCGGACAAGTGCAGCTC
CTGGAGTCTGGGGCTGAGCTGGTGAGGGGTGGAGGCGGTTCCCATCACCATCACCATCACTGATGAAGATCTCAAT
TGGAATTCTGA
SEQ ID NO: 26; 401 AA
MDPRCTMGLAILIFVTVLLISDAVSVETQAYFNGTAYLPCPFTKAQNISLSELVVFWQDQQKLVLYEHYLGTEKLD
SVNAKYLGRTSFDRNNWTLRLHNVQIKDMGSYDCFIQKKPPTGSIILQQTLTELSVIANFSEPEIKLAQNVTGNSG
INLTCTSKQGHPKPKKMYFLITNSTNEYGDNMQISQDNVTELFSISNSLSLSFPDGVWHMTVVCVLETESMKISSK
PLNFTQEFPSPQTYWKEAAAGSSVKISCKASGYAFSSYWMNWVKQRPGQGLEWIGQIWPGDGDTNYNGKFKGKATL
TADESSSTAYMQLSSLRSEDSAVYSCARRETTTVGRYYYAMDYWGQGTTVTSPGSGGGGSGGGGSGGGGSSGQVQL
LESGAELVRGGGGSHHHHHH**
sCD86-ABLoop Open hCD19LC Altlg SEQ ID NO: 27; 1200 NT
ATGGACCCCAGATGCACCATGGGCTTGGCAATCCTTATCTTTGTGACAGTCTTGCTGATCTCAGATGCTGTTTCCG
TGGAGACGCAAGCTTATTTCAATGGGACTGCATATCTGCCGTGCCCATTTACAAAGGCTCAAAACATAAGCCTGAG
TGAGCTGGTAGTATTTTGGCAGGACCAGCAAAAGTTGGTTCTGTACGAGCACTATTTGGGCACAGAGAAACTTGAT
AGTGTGAATGCCAAGTACCTGGGCCGCACGAGCTTTGACAGGAACAACTGGACTCTACGACTTCACAATGTTCAGA
TCAAGGACATGGGCTCGTATGATTGTTTTATACAAAAAAAGCCACCCACAGGATCAATTATCCTCCAACAGACATT
AACAGAACTGTCAGTGATCGCCAACTTCAGTGAACCTGAAATAAAACTGGCTCAGAATGTAACAGGAAATTCTGGC
ATAAATTTGACCTGCACGTCTAAGCAAGGTCACCCGAAACCTAAGAAGATGTATTTTCTGATAACTAATTCAACTA
ATGAGTATGGTGATAACATGCAGATATCACAAGATAATGTCACAGAACTGTTCAGTATCTCCAACAGCCTCTCTCT
TTCATTCCCGGATGGTGTGTGGCATATGACCGTTGTGTGTGTTCTGGAAACGGAGTCAATGAAGATTTCCTCCAAA
CCTCTCAATTTCACTCAAGAGTTTCCATCTCCTCAAACGTATTGGAAGGAGGCGGCCGCAGGGCAGAGGGCCACCA
TCTCCTGCAAGGCCAGCCAAAGTGTTGATTATGATGGTGATAGTTATTTGAACTGGTACCAACAGATTCCAGGACA
GCCACCCAAACTCCTCATCTATGATGCATCCAATCTAGTTTCTGGGATCCCACCCAGGTTTAGTGGCAGTGGGTCT
GGGACAGACTTCACCCTCAACATCCATCCTGTGGAGAAGGTGGATGCTGCAACCTATCACTGTCAGCAAAGTACTG
AGGATCCGTGGACGTTCGGTGGAGGGACCAAGCTGGAAATAAAATCTCCTGGATCCGGTGGAGGCGGATCAGGTGG
CGGTGGAAGTGGAGGTGGTGGATCTTCCGGAGAGCTCGTGCTCACCCAGTCTCCAGCTTCTTTGGCTGTGTCTCTA
GGTGGAGGCGGTTCCCATCACCATCACCATCACTGATGAAGATCTCAATTGGAATTCTGA
SEQ ID NO: 28; 392 AA
MDPRCTMGLAILIFVTVLLISDAVSVETQAYFNGTAYLPCPFTKAQNISLSELVVFWQDQQKLVLYEHYLGTEKLD
SVNAKYLGRTSFDRNNWTLRLHNVQIKDMGSYDCFIQKKPPTGSIILQQTLTELSVIANFSEPEIKLAQNVTGNSG
INLTCTSKQGHPKPKKMYFLITNSTNEYGDNMQISQDNVTELFSISNSLSLSFPDGVWHMTVVCVLETESMKISSK
PLNFTQEFPSPQTYWKEAAAGQRATISCKASQSVDYDGDSYLNWYQQIPGQPPKLLIYDASNLVSGIPPRFSGSGS
GTDFTLNIHPVEKVDAATYHCQQSTEDPWTFGGGTKLEIKSPGSGGGGSGGGGSGGGGSSGELVLTQSPASLAVSL
GGGGSHHHHHH**

### Polypeptide Linker Sequences

Polypeptide linkers can be used to join the N- and C-termini of the immunoglobulin domains and join fusion proteins attached to the new N′- and C′-termini. The linker joining the N- and C-termini can comprise from about 15 to about 30 amino acid residues. Typically, the linker is about 20-30 amino acid residues (e.g., 18-32) and comprises a poly-glycine, poly-alanine, or poly-glycine-serine linker (e.g., SEQ ID NO: 35-47). Internal linkers of 3-6 amino acid residues may be used between fused domains (e.g., SEQ ID NO: 48-52). Exemplary linker nucleotide and polypeptide sequences are shown:

SEQ ID NO: 35; 60 NT
cctggatccggtggaggcggatcaggtggcggtggaagtggaggtggtgg
atcttccgga
SEQ ID NO: 36; 20 AA
PGSGGGGSGGGGSGGGGSSG
SEQ ID NO: 37; 57 NT
ggatccggtggaggcggatcaggtggcggtggaagtggaggtggtggatc
ttccgga
SEQ ID NO: 38; 19 AA
GSGGGGSGGGGSGGGGSSG
SEQ ID NO: 39; 75 NT
cctggatccggtggaggcggatcaggtggcggtggaagtggaggtggtgg
atctggcggaggcggatcttccgga
SEQ ID NO: 40; 25 AA
PGSGGGGSGGGGSGGGGSGGGGSSG
SEQ ID NO: 41; 72 NT
ggatccggtggaggcggatcaggtggcggtggaagtggaggtggtggatc
tggcggaggcggatcttccgga
SEQ ID NO: 42; 24 AA
GSGGGGSGGGGSGGGGSGGGGSSG
SEQ ID NO: 43; 90 NT
cctggatccggtggaggcggatcaggtggcggtggaagtggaggtggtgg
atctggcggaggcggatctggaggtggaggctcttccgga
SEQ ID NO: 44; 30 AA
PGSGGGGSGGGGSGGGGSGGGGSGGGGSSG
SEQ ID NO: 45; 87 NT
ggatccggtggaggcggatcaggtggcggtggaagtggaggtggtggatc
tggcggaggcggatctggaggtggaggctcttccgga
SEQ ID NO: 46; 29 AA
GSGGGGSGGGGSGGGGSGGGGSGGGGSSG
SEQ ID NO: 47; 18 NT
ggaggttccgcggccgca
SEQ ID NO: 48; 6 AA
GGSAAA
SEQ ID NO: 49; 15 NT
ggtggaggcggttcc
SEQ ID NO: 50; 5 AA
GGGGS
SEQ ID NO: 51; 9 NT
gcggccgca
SEQ ID NO: 52; 3 AA
AAA
SEQ ID NO: 53; 12 NT
cctggatccggt
SEQ ID NO: 54; 4 AA
PGSG
SEQ ID NO: 55; 18 NT
ggtcctggatccggtgga
SEQ ID NO: 56; 6 AA
GPGSGG
SEQ ID NO: 57; 24 NT
ggtggacctggatccggtggaggc
SEQ ID NO: 58; 8 AA
GGPGSGGG

mMHCII-Anti-hCD19 BMiMS

The moth cytochrome C peptide 88-93 (MCC) presented in the mouse MHCII 1-Ek (MCC:I-E^{k}) was utilized. The gene encoding the MHCIIa (I-E^{k}a) fused to the anti-hCD19 AltIg LC was subcloned into the “pZ4” zeocin-resistance MSCV vector (MCS-IRES-Zeo resistance [1]). The gene encoding the MHCIIb (MCC:I-E^{k}b) fused to the anti-hCD19 AltIg HC was subcloned into the “pP2” puromycin-resistance MSCV vector (MCS-IRES-Puro [1]).

mCD80-Anti-hCD19 BMiMS (Note that CD80 is a Homodimer)

The gene encoding mCD80 fused to the anti-hCD19 AltIg HC was subcloned into the “pP2” puromycin-resistance MSCV vector (MCS-IRES-Puro resistance [1]). The gene encoding mCD80 fused to the anti-hCD19 AltIg LC was subcloned into the “pZ4” zeocin-resistance MSCV vector (MCS-IRES-Zeo [1]).

mCD86-Anti-hCD19 BMiMS (Note that CD86 is a Monomer)

The gene encoding the anti-hCD19 AltIg LC was subcloned into the “pZ4” zeocin-resistance MSCV vector (MCS-IRES-Zeo resistance [1]). The gene encoding mCD86 fused to the anti-hCD19 AltIg HC was subcloned into the “pP2” puromycin-resistance MSCV vector (MCS-IRES-Puro (1)).

mICAM-1-Anti-hCD19 BMiMS (Note that ICAM-1 is a Monomer).

The gene encoding the anti-hCD19 AltIg LC was subcloned into the “pZ4” zeocin-resistance MSCV vector (MCS-IRES-Zeo resistance [1]). The gene encoding mICAM-1 fused to the anti-hCD19 AltIg HC was subcloned into the “pP2” puromycin-resistance MSCV vector (MCS-IRES-Puro (1)).

### Example 2

### Retroviral Transduction

M12 B cell lymphoma cell lines were generated as described previously [2]. In brief, for each construct 1.3×10⁶ Phoenix E packaging cells were plated in complete DMEM (10% FCS) and cultured overnight at 37° C. in a 6 cm plate (Falcon). The media was exchanged, and the cells were transfected with 1.5 μg of the desired retroviral construct using Turbofect (Fermentas) according to the manufacturer's instructions. The media was changed after 24 hrs and the cells were shifted to 32° C. The viral supernatant was then harvested at 48 and 72 hrs. The supernatant for all constructs used to generate a cell line (e.g., AltIg LC plus AltIg HC) were then pooled and concentrated to 250 μL using an Amicon Ultra 15 100 kDa (Millipore). 1×10⁶ parental M12 cells were then plated in 2 mL of complete RPMI (5% FCS) in one well of a 12 well plate in 4 μg/mL polybrene plus the viral supernatant and spun for 2 hrs at 32° C. at 2700 rpm in a Legend XTR centrifuge (ThermoFisher). The media was exchanged immediately after spin infection and the cells were cultured overnight at 37° C. prior to selection with 10 μg/mL puromycin (LifeTech) and 100 μg/mL Zeocin (LifeTech) splitting as necessary to keep thin and under heavy selection. Drug concentrations were reduced to 5 μg/mL puromycin and 50 μg/mL Zeocin on day 5-7 after selection for maintenance.

### Example 3

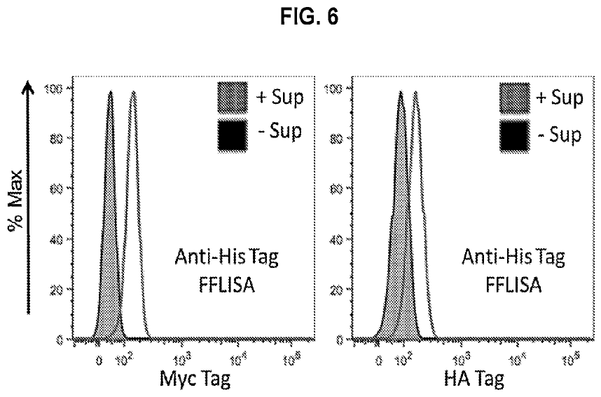

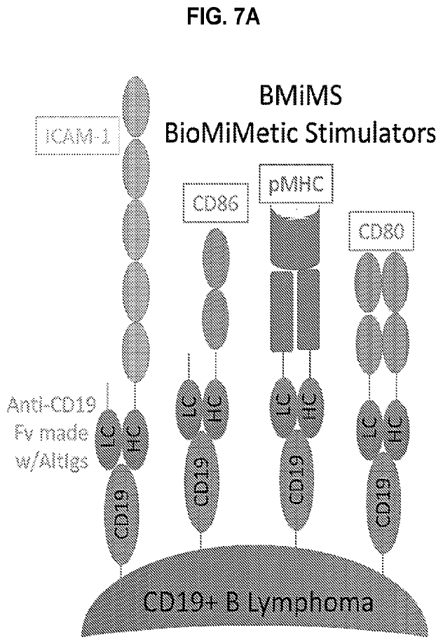

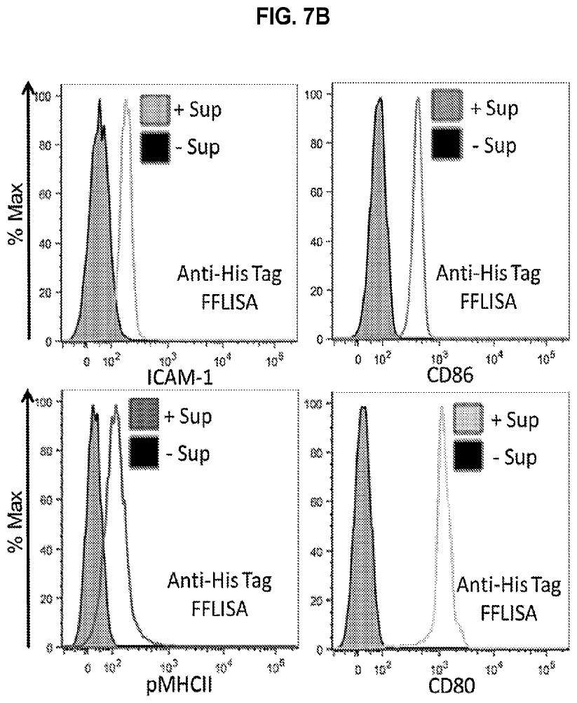

AltIg with Opened C-C′ Loop Flow-based Fuorophore-linked Immunosorbent Assay (FFLISA) M12 cells were cultured to confluency (1-2×10⁶ cells/mL) in complete RPMI (5% FCS) and the supernatant was harvested for analysis. 6.0 μm streptavidin-coated polystyrene microspheres (Polysciences) were further coated with biotinylated anti-His Tag antibody (clone HIS.H8, Invitrogen), washed, and incubated with media (negative control) or 0.250 mL of concentrated (50 mL down to 0.250 mL in an Amicon Ultra 15 10 kDa (Millipore)) M12 cell culture supernatant at 4° C. for 1 hour. After washing, beads were probed with Alexa Flour 594 conjugated anti-mouse Myc tag (clone 9E10, BioLegend), Alexa Flour 488 conjugated anti-mouse HA tag (clone 16B12, BioLegend), Alexa Flour 647 conjugated anti-mouse anti-I-Ek (clone 14-4-4s, BioLegend), PE conjugated anti-mCD80 (clone 16-10A1, BioLegend), anti-mCD86 (clone GL-1, BioLegend), or anti-mICAM-1 (clone YN1/1.7.4, eBioscience) and analyzed by flow cytometry [3]. Results are shown in FIG. 6 and FIG. 7B

### Example 4

Ramos Cell Staining with BMiMS

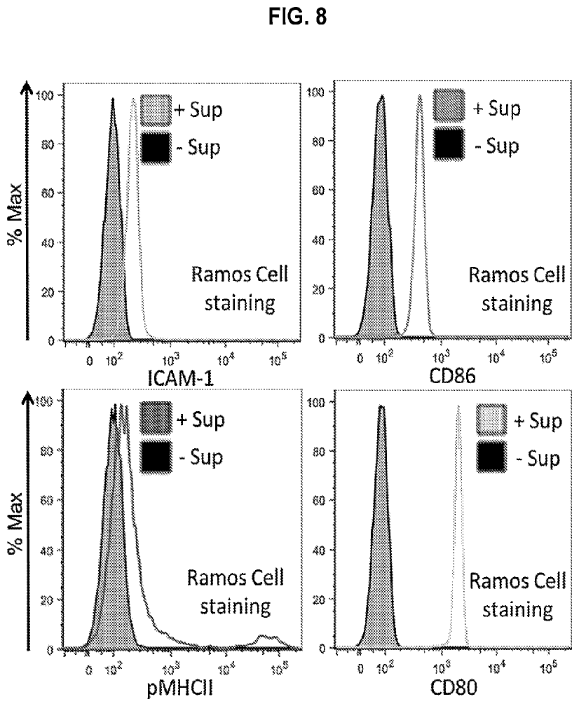

M12 cells were cultured to confluency (1-2×10⁶ cells/mL) in complete RPMI (5% FCS) and the supernatant was harvested for analysis as described above. Ramos cells were harvested, washed, and 1×10⁶ were incubated with media (negative control) or 0.250 mL of concentrated (50 mL down to 0.250 mL in Amicon Ultra 15 10 kDa (Millipore)) M12 cell culture supernatant at 4° C. for 1 hour. After washing, cells were stained with PE conjugated anti-mouse I-A/I-E (clone M5/14. 15.2, eBiosciences), PE conjugated anti-mCD80 (clone 16-10A1, BioLegend), anti-mCD86 (clone GL-1, BioLegend) or anti-mlCAM-1 (clone YN1/1.7.4, eBioscience) and analyzed by flow cytometry [3]. Results are shown in FIG. 8.

### Example 5

Stimulation of Naive CD4+ T Cells with BMiMS

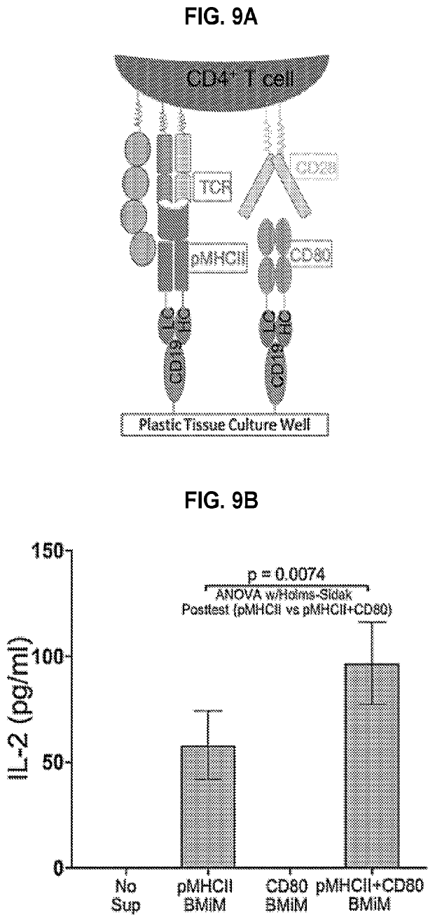

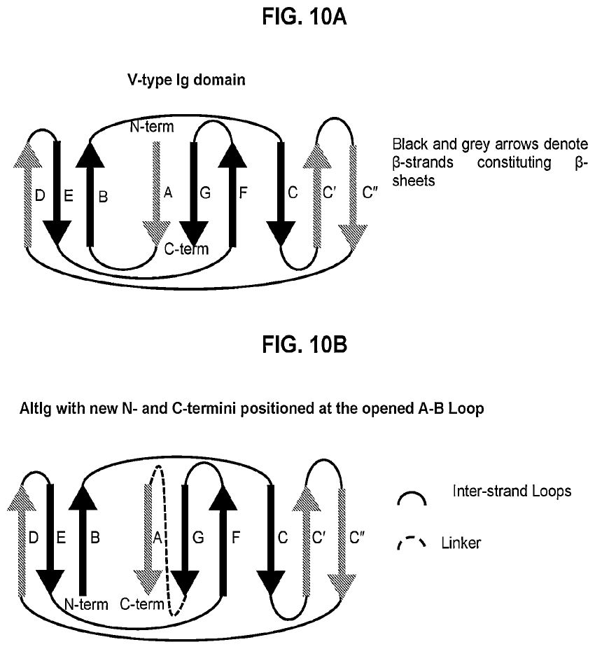

An experiment was designed to determine whether BMiMS can bind huCD19 and stimulate CD4+ T cells. See FIG. 9A. pMHCII-based BMiMS were made with the moth cytochrome c 88-93 peptide (MCC) tethered with a flexible linker to I-E^{k} (MCC:I-E^{k}) which is recognized by the 5c.c7 TCR. For a pilot experiment, the wells of a 96-well plate were coated with 4 μg/ml recombinant huCD19 (BioLegend). The wells were washed and then incubated with concentrated supernatant from cells expressing pMHCII-BMiMS, CD80-BMiMS, or both. Naive 5c.c7+CD4+ T cells from 5c.c7 TCR transgenic mice (RagKO) were then incubated for 18 hr in the wells prior to harvesting of the supernatant for measurement of IL-2 by ELISA. No IL-2 was produced by cells cultured in wells with huCD19 only or the huCD19+CD80 BMiMS. IL-2 was produced by T cells cultured in wells with huCD19+pMHCII-BMiMS, and higher levels of IL-2 were produced by T cells cultured in wells coated with huCD19+pMHCII-BMiMS+CD80-BMiMS. These data provide evidence that BMiMS bound to CD19 can stimulate CD4+ T cells. See FIG. 9B.

### Example 6

AltIg with Opened A-B Loop FFLISA

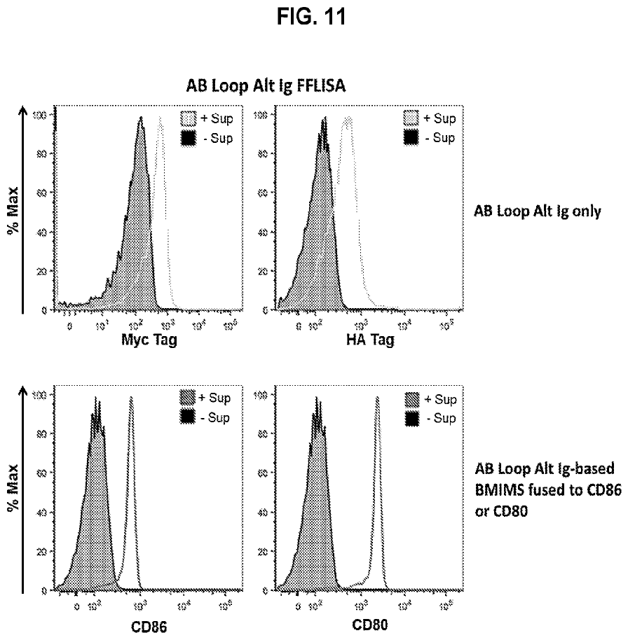

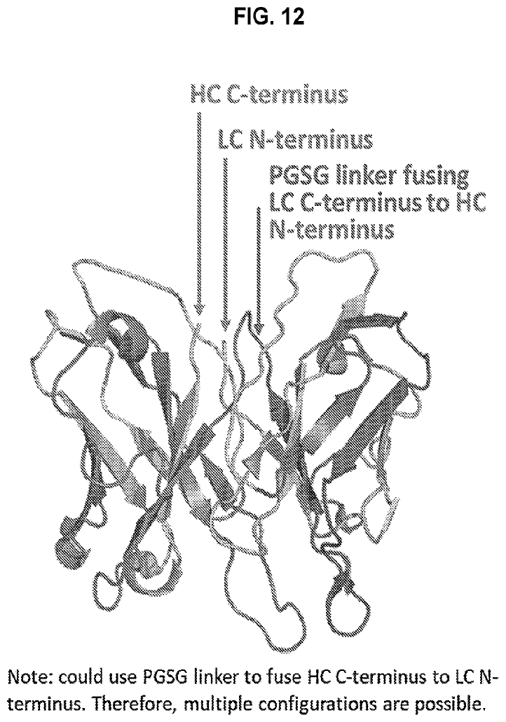

M12 cells were cultured to confluency (1-2×10⁶ cells/mL) in complete RPMI (5% FCS) and the supernatant was harvested for analysis. 6.0 μm streptavidin-coated polystyrene microspheres (Polysciences) were further coated with biotinylated anti-His Tag antibody (clone HIS.H8, Invitrogen), washed, and incubated with media (negative control) or 0.250 mL of concentrated (50 mL down to 0.250 mL in an Amicon Ultra 15 10 kDa (Millipore)) M12 cell culture supernatant at 4° C. for 1 hour. After washing, beads were probed with Alexa Flour 594 conjugated anti-mouse Myc tag (clone 9E10, BioLegend), Alexa Flour 488 conjugated anti-mouse HA tag (clone 16B12, BioLegend), PE conjugated anti-mCD80 (clone 16-10A1, BioLegend), or PE conjugated anti-mCD86 (clone GL-1, BioLegend) and analyzed by flow cytometry [3]. Results are shown in FIG. 11.

### Example 7

### AltIg-scFv and Bivalent AltIg-scFvs

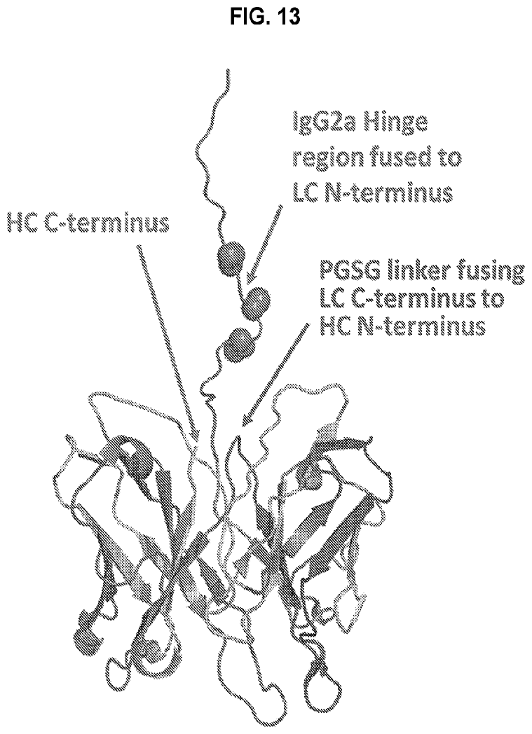

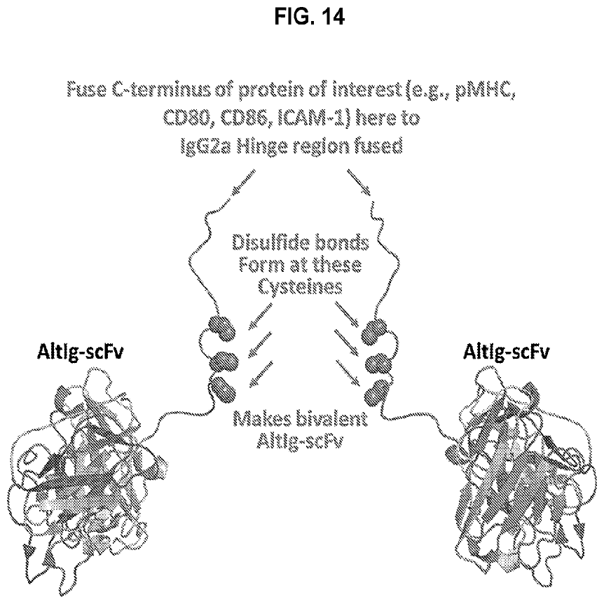

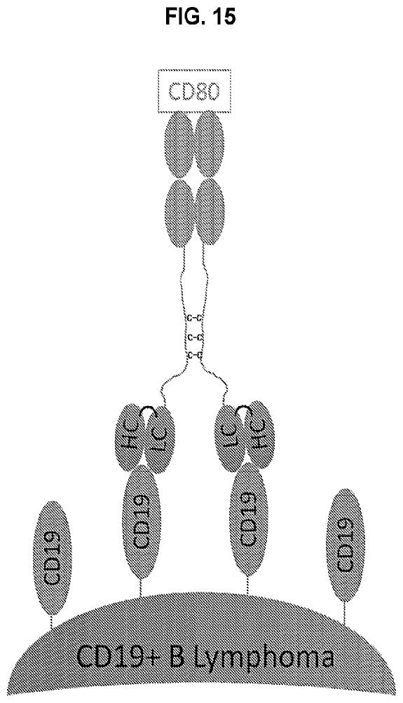

AltIg-scFv and Bivalent AltIg-scFvs constructs will be generated using standard molecular biology techniques. FIG. 13-14 illustrate how AltIgs can be used to construct scFvs or bivalent AltIg-ScFvs. For example, a hinge region from an antibody (e.g., a mouse IgG2a or human IgG2a) can be fused to an AltIg-scFv with an optional liker. The cysteines in the hinge region are capable of forming disulfide bonds and can create novel bivalent molecules. Fusing this to other molecules of interest (e.g. pMHCII, CD80, CD86, ICAM-1) could be done to make bivalent BMiMS.

Exemplary constructs and their nucleotide (cDNA) and amino acid sequences are shown. The lowercase characters indicate inserted nucleotides or the linker regions in the polypeptide sequence.

anti-hCD19 Altlg-scFv.IgHinge SEQ ID NO: 29; 963 NT
TCCGGAGGGCCCACAATCAAGCCCTGTCCTCCATGCAAATGCCCAGCACCTAACCTCTTGGGTGGAGGATCCGGCC
AGCCACCCAAACTCCTCATCTATGATGCATCCAATCTAGTTTCTGGGATTCCACCCAGGTTTAGTGGCAGTGGGTC
TGGGACAGACTTCACCCTCAACATCCATCCTGTGGAGAAGGTGGATGCTGCAACCTATCACTGTCAGCAAAGTACT
GAAGATCCGTGGACGTTCGGTGGAGGGACCAAGCTGGAAATAAAATCTcccggttcaggaggaggtggctccggtg
gaggagggtctggcggaggaggctcaagcggaGAGCTCGTGCTCACCCAGTCTCCAGCTTCTTTGGCTGTGTCTCT
AGGGCAGAGGGCCACCATCTCCTGCAAGGCCAGCCAAAGTGTTGATTATGATGGTGATAGTTATTTGAACTGGTAC
CAACAGATTCCAGGAAGCGGCTCTGGACAGGGTCTTGAGTGGATTGGACAGATTTGGCCTGGAGATGGTGATACTA
ACTACAATGGAAAGTTCAAGGGTAAAGCCACTCTGACTGCAGACGAATCCTCCAGCACAGCCTACATGCAACTCAG
CAGCCTACGATCTGAGGACTCTGCGGTCTATTCTTGTGCAAGACGGGAGACTACGACGGTAGGCCGTTATTACTAT
GCTATGGACTACTGGGGCCAAGGGACCACGGTCACCTCTccagggagtggcggcggcggatccggaggaggcgggt
caggtggcggcggctcatctgggCAAGTGCAGCTCCTGGAGTCTGGGGCTGAGCTGGTGAGGCCTGGGTCCTCAGT
GAAGATTTCCTGCAAGGCTTCTGGCTATGCATTCAGTAGCTACTGGATGAACTGGGTGAAGCAGAGGGGTGGAGGC
GGTTCCCATCACCATCACCATCACTGATGAAGATCTCAATTGGAATTCTGA
SEQ ID NO: 30, 313 AA
SGGPTIKPCPPCKCPAPNLLGGGSGQPPKLLIYDASNLVSGIPPRFSGSGSGTDFTLNIHPVEKVDAATYHCQQST
EDPWTFGGGTKLEIKSPGSGGGGSGGGGSGGGGSSGELVLTQSPASLAVSLGQRATISCKASQSVDYDGDSYLNWY
QQIPGSGSGQGLEWIGQIWPGDGDTNYNGKFKGKATLTADESSSTAYMQLSSLRSEDSAVYSCARRETTTVGRYYY
AMDYWGQGTTVTSPGSGGGGSGGGGSGGGGSSGQVQLLESGAELVRPGSSVKISCKASGYAFSSYWMNWVKQRGGG
GSHHHHHH**
sCD80-IgHinge.hCD19 Altlg LCHC SEQ ID NO: 31; 1713 NT
ATGGCTTGCAACTGTCAGTTGATGCAGGATACACCACTCCTCAAGTTTCCATGTCCAAGGCTCATTCTTCTCTTTG
TGCTGCTGATTCGTCTTTCACAAGTGTCTTCAGATGTTGATGAACAACTGTCCAAGTCAGTGAAAGATAAGGTATT
GCTGCCTTGCCGTTACAACTCTCCTCATGAAGATGAGTCTGAAGACCGAATCTACTGGCAAAAACATGACAAAGTG
GTGCTGTCTGTCATTGCTGGGAAACTAAAAGTGTGGCCCGAGTATAAGAACCGGACTTTATATGACAACACTACCT
ACTCTCTTATCATCCTGGGCCTGGTCCTTTCAGACCGGGGCACATACAGCTGTGTCGTTCAAAAGAAGGAAAGAGG
AACGTATGAAGTTAAACACTTGGCTTTAGTAAAGTTGTCCATCAAAGCTGACTTCTCTACCCCCAACATAACTGAG
TCTGGAAACCCATCTGCAGACACTAAAAGGATTACCTGCTTTGCTTCCGGGGGTTTCCCAAAGCCTCGCTTCTCTT
GGTTGGAAAATGGAAGAGAATTACCTGGCATCAATACGACAATTTCCCAAGATCCTGAATCTGAATTGTACACCAT
TAGTAGCCAACTAGATTTCAATACGACTCGCAACCACACCATTAAGTGTCTCATTAAATATGGAGATGCTCACGTG
TCAGAGGACTTCACCTGGGAAAAACCCCCAGAAGACCCTCCTGATAGCAAGAACACAGCGGCCGCATCCGGAGGGC
CCACAATCAAGCCCTGTCCTCCATGCAAATGCCCAGCACCTAACCTCTTGGGTGGAGGATCCGGCCAGCCACCCAA
ACTCCTCATCTATGATGCATCCAATCTAGTTTCTGGGATTCCACCCAGGTTTAGTGGCAGTGGGTCTGGGACAGAC
TTCACCCTCAACATCCATCCTGTGGAGAAGGTGGATGCTGCAACCTATCACTGTCAGCAAAGTACTGAAGATCCGT
GGACGTTCGGTGGAGGGACCAAGCTGGAAATAAAATCTcccggttcaggaggaggtggctccggtggaggagggtc
tggcggaggaggctcaagcggaGAGCTCGTGCTCACCCAGTCTCCAGCTTCTTTGGCTGTGTCTCTAGGGCAGAGG
GCCACCATCTCCTGCAAGGCCAGCCAAAGTGTTGATTATGATGGTGATAGTTATTTGAACTGGTACCAACAGATTC
CAGGAAGCGGCTCTGGACAGGGTCTTGAGTGGATTGGACAGATTTGGCCTGGAGATGGTGATACTAACTACAATGG
AAAGTTCAAGGGTAAAGCCACTCTGACTGCAGACGAATCCTCCAGCACAGCCTACATGCAACTCAGCAGCCTACGA
TCTGAGGACTCTGCGGTCTATTCTTGTGCAAGACGGGAGACTACGACGGTAGGCCGTTATTACTATGCTATGGACT
ACTGGGGCCAAGGGACCACGGTCACCTCTccagggagtggcggcggcggatccggaggaggcgggtcaggtggcgg
cggctcatctgggCAAGTGCAGCTCCTGGAGTCTGGGGCTGAGCTGGTGAGGCCTGGGTCCTCAGTGAAGATTTCC
TGCAAGGCTTCTGGCTATGCATTCAGTAGCTACTGGATGAACTGGGTGAAGCAGAGGGGTGGAGGCGGTTCCCATC
ACCATCACCATCACTGATGAAGATCTCAATTGGAATTCTGA
SEQ ID NO: 32, 563 AA
MACNCQLMQDTPLLKFPCPRLILLFVLLIRLSQVSSDVDEQLSKSVKDKVLLPCRYNSPHEDESEDRIYWQKHDKV
VLSVIAGKLKVWPEYKNRTLYDNTTYSLIILGLVLSDRGTYSCVVQKKERGTYEVKHLALVKLSIKADFSTPNITE
SGNPSADTKRITCFASGGFPKPRFSWLENGRELPGINTTISQDPESELYTISSQLDFNTTRNHTIKCLIKYGDAHV
SEDFTWEKPPEDPPDSKNTAAASGGPTIKPCPPCKCPAPNLLGGGSGQPPKLLIYDASNLVSGIPPRFSGSGSGTD
FTLNIHPVEKVDAATYHCQQSTEDPWTFGGGTKLEIKSPGSGGGGSGGGGSGGGGSSGELVLTQSPASLAVSLGQR
ATISCKASQSVDYDGDSYLNWYQQIPGSGSGQGLEWIGQIWPGDGDTNYNGKFKGKATLTADESSSTAYMQLSSLR
SEDSAVYSCARRETTTVGRYYYAMDYWGQGTTVTSPGSGGGGSGGGGSGGGGSSGQVQLLESGAELVRPGSSVKIS
CKASGYAFSSYWMNWVKQRGGGGSHHHHHH**
anti-hCD19 Altlg-scFv SEQ ID NO: 33; 888 NT
CAGCCACCCAAACTCCTCATCTATGATGCATCCAATCTAGTTTCTGGGATTCCACCCAGGTTTAGTGGCAGTGGGT
CTGGGACAGACTTCACCCTCAACATCCATCCTGTGGAGAAGGTGGATGCTGCAACCTATCACTGTCAGCAAAGTAC
TGAAGATCCGTGGACGTTCGGTGGAGGGACCAAGCTGGAAATAAAATCTcccggttcaggaggaggtggctccggt
ggaggagggtctggcggaggaggctcaagcggaGAGCTCGTGCTCACCCAGTCTCCAGCTTCTTTGGCTGTGTCTC
TAGGGCAGAGGGCCACCATCTCCTGCAAGGCCAGCCAAAGTGTTGATTATGATGGTGATAGTTATTTGAACTGGTA
CCAACAGATTCCAGGAAGCGGCTCTGGACAGGGTCTTGAGTGGATTGGACAGATTTGGCCTGGAGATGGTGATACT
AACTACAATGGAAAGTTCAAGGGTAAAGCCACTCTGACTGCAGACGAATCCTCCAGCACAGCCTACATGCAACTCA
GCAGCCTACGATCTGAGGACTCTGCGGTCTATTCTTGTGCAAGACGGGAGACTACGACGGTAGGCCGTTATTACTA
TGCTATGGACTACTGGGGCCAAGGGACCACGGTCACCTCTccagggagtggcggcggcggatccggaggaggcggg
tcaggtggcggcggctcatctgggCAAGTGCAGCTCCTGGAGTCTGGGGCTGAGCTGGTGAGGCCTGGGTCCTCAG
TGAAGATTTCCTGCAAGGCTTCTGGCTATGCATTCAGTAGCTACTGGATGAACTGGGTGAAGCAGAGGGGTGGAGG
CGGTTCCCATCACCATCACCATCACTGATGAAGATCTCAATTGGAATTCTGA
SEQ ID NO: 34; 288 AA
QPPKLLIYDASNLVSGIPPRFSGSGSGTDFTLNIHPVEKVDAATYHCQQSTEDPWTFGGGTKLEIKSPGSGGGGSG
GGGSGGGGSSGELVLTQSPASLAVSLGQRATISCKASQSVDYDGDSYLNWYQQIPGSGSGQGLEWIGQIWPGDGDT
NYNGKFKGKATLTADESSSTAYMQLSSLRSEDSAVYSCARRETTTVGRYYYAMDYWGQGTTVTSPGSGGGGSGGGG
SGGGGSSGQVQLLESGAELVRPGSSVKISCKASGYAFSSYWMNWVKQRGGGGSHHHHHH**

### REFERENCES

1. Kuhns and Davis, “Disruption of extracellular interactions impairs T cell receptor-CD3 complex stability and signaling,” Immunity 26: 357-369 (2007).
2. Lee et al., “A Mechanical Switch Couples T Cell Receptor Triggering to the Cytoplasmic Juxtamembrane Regions of CD3zetazeta,” Immunity 43: L 227-239 (2015).
3. Glassman et al., “The CD4 and CD3deltaepsilon Cytosolic Juxtamembrane Regions Are Proximal within a Compact TCR-CD3-pMHC-CD4 Macrocomplex,” J. Immunol. 196: 4713-4722 (2016).

What is claimed:

1. A nucleotide sequence encoding a polypeptide, where the polypeptide comprises one or more immunoglobulin domains comprising a fusion of the wild type N- and C-termini or an optional a linker joining the wild type N- and C-termini and a scission within one of the loop regions yielding novel N′- and C′-termini.

2. The nucleotide sequence of claim 1, wherein the immunoglobulin domain comprises an immunoglobulin domain from an immunoglobulin, Fab, Fv, T cell receptor (TCR), CD80, CTLA-4, PD1, PDL1, MHC molecules, or other immunoglobulin domain containing proteins.

3. The nucleotide sequence of claim 1 or 2, wherein the immunoglobulin domain comprises a heavy chain variable domain or a light chain variable domain.

4. The nucleotide sequence of any one of claims 1-3, wherein one or both of the N′- and C′-termini are fused with one or more additional polypeptides.

5. The nucleotide sequence of any one of claims 1-4, wherein the additional polypeptide comprises an immunoglobulin domain, Fab, Fv, ScFV, cell receptor, pMHC, costimulatory molecule, cytokine, or another polypeptide domain.

6. The nucleotide sequence of any one of claims 1-4, wherein the additional polypeptide comprises an immunoglobulin hinge region.

7. The nucleotide sequence of any one of claims 1-4, wherein the additional polypeptide comprises one or more of: CD80, CD86, ICAM-1, PD-L1/L2, B7H1, B7H2, CD40, CD40L, CD47, CD48, CD58, 4-1BBL, OX40L, TIM-1, TIM-4, CD80:PD-L1 heterodimer, calreticulin, a peptide that is at least 90% identical to CD80, CD86, ICAM-1, PD-L1/L2, B7H1, B7H2, CD40, CD40L, CD47, CD48, CD58, 4-1BBL, OX40L, TIM-1, TIM-4, CD80:PD-L1 heterodimer, calreticulin, fragments thereof, or combinations thereof; cytokines: IFNα, IFNβ, IFNγ, IL-1, IL-1α, IL-2, IL-4, IL-6, IL-7, IL-10, IL-12, IL-15, IL-21, IL-23, TNF, TNFα, TGFβ, GM-CSF, CSF-1, a peptide that is at least 90% identical to IFNα, IFNβ, IFNγ, IL-1, IL-1α, IL-2, IL-4, IL-6, IL-7, IL-10, IL-12, IL-15, IL-21, IL-23, TNF, TNFα, TGFβ, GM-CSF, CSF-1, fragments thereof, or combinations thereof; MHC alleles: MHC molecule comprises HLA-A, HLA-B, HLA-C, β2-microglobulin, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ, a peptide that is at least 90% identical to HLA-A, HLA-B, HLA-C, HLA-DPA1, HLA-DPB1, HLA-DQA1, HLA-DQB1, HLA-DRA, HLA-DRB, H2-Aa, H2-B1, H2-K1, H2-EB β, H2-EKα, H2-EKβ, fragments thereof, or combinations thereof; or TCR molecules: TRAC, TRBC1, TRBC2, TRDC, TRGC1, TRGC2, TCRA, TCB1, TCB2, TCC1, TCC2, TCC3, TCC4, a peptide that is at least 90% identical to TRAC, TRBC1, TRBC2, TRDC, TRGC1, TRGC2, TCRA, TCB1, TCB2, TCC1, TCC2, TCC3, TCC4, fragments thereof, or combinations thereof.

8. The nucleotide sequence of any one of claims 1-7, wherein the optional linker comprises a polypeptide linker or a chemical linker.

9. The nucleotide sequence of any one of claims 1-8, wherein the I optional inker comprises a polypeptide selected from one or more of a poly glycine linker, poly alanine linker, poly glycine-alanine linker, poly glycine-serine linker, or poly glycine-serine-proline linker.

10. The nucleotide sequence of any one of claims 1-9, wherein the optional linker comprises a polypeptide having 85% to 99% identity to one or more of SEQ ID NO: 36, 38, 40, 42, 44, 46, 48, 50, 52, 54, 56, or 58.

11. The nucleotide sequence of any one of claims 1-10, wherein the optional linker is a polypeptide selected from one or more of SEQ ID NO: 36, 38, 40, 42, 44, 46, 48, 50, 52, 54, 56, or 58.

12. The nucleotide sequence of any one of claims 1-11, wherein the loop region where scission occurs comprises one or more of the loops connecting adjacent β-strands comprising A-B, B-C, C-C′, C′-C″, C-D, C′-D, C″-D, D-E, E-F, F-G, or other loop-linkages that eliminate one or more intervening β-strands.

13. The nucleotide sequence of any one of claims 1-12, wherein the loop region where scission occurs comprises one or more of the immunoglobulin β-sheet loops A-B, B-C, C-C′, C′-C″, C-D, C′-D, C″-D, D-E, E-F, or F-G.

14. The nucleotide sequence of any one of claims 1-13, wherein the loop region where scission occurs comprises the C-C′ or A-B loop.

15. The nucleotide sequence of any one of claims 1-14, wherein the nucleotide sequence has 85% to 99% identity to SEQ ID NO: 1, 3, 5, 7, 9, 11, 13, 15, 17, 19, 21, 23, 25, 27, 29, 31, or 33.

16. The nucleotide sequence of any one of claims 1-15, wherein the nucleotide sequence is selected from SEQ ID NO: 1, 3, 5, 7, 9, 11, 13, 15, 17, 19, 21, 23, 25, 27, 29, 31, or 33.

17. A polynucleotide vector comprising one or more nucleotide sequences of any one of claims 1-16.

18. A cell comprising one or more nucleotide sequences of claim 1 or a polynucleotide vector of claim 17.

19. A polypeptide encoded by the nucleotide sequence of any one of claims 1-16.

20. A polypeptide encoded by the nucleotide sequence of any one of claims 1-16, wherein the polypeptide has 85% to 99% identity to SEQ ID NO: 2, 4, 6, 8, 10, 12, 14, 16, 18, 20, 22, 24, 26, 28, 30, 32, or 34.

21. A polypeptide encoded by the nucleotide sequence of any one of claims 1-16, wherein the polypeptide is selected from SEQ ID NO: 2, 4, 6, 8, 10, 12, 14, 16, 18, 20, 22, 24, 26, 28, 30, 32, or 34.

22. A bivalent polypeptide complex comprising a dimer of the polypeptides of SEQ ID NO: 32 or 34 covalently linked via one or more disulfide bonds.

23. A single chain variable fragment (scFv) polypeptide comprising SEQ ID NO: 34.

24. A process for manufacturing the nucleotide sequence of any one of claims 1-16 or a polypeptide encoded by the nucleotide sequence of any one of claims 1-16, the process comprising: transforming or transfecting a cell with the nucleic acid; growing the cells; optionally isolating additional quantities of the nucleotide sequence; inducing expression of the polypeptide; and isolating the polypeptide.

25. A means for manufacturing the nucleotide sequence of any one of claims 1-16 or a polypeptide encoded by the nucleotide sequence of any one of claims 1-16, the means comprising: transforming or transfecting a cell with the nucleic acid; growing the cells; optionally isolating additional quantities of the nucleotide sequence; inducing expression of the polypeptide; and isolating the polypeptide.

26. The nucleotide sequence of any one of claims 1-16 or a polypeptide encoded by the nucleotide sequence of any one of claims 1-16 produced by the process of claim 21 or the means of claim 22.

27. A method of treatment comprising administering an effective amount of polypeptide encoded by the nucleotide sequence of any one of claims 1-16 to a subject in need thereof.

28. Use of an effective amount of a polypeptide encoded by the nucleotide sequence of any one of claims 1-16 for the treatment of a disease or disorder comprising administering an effective amount the polypeptide to a subject in need thereof.

29. A research tool comprising a polypeptide encoded by the nucleotide sequence of any one of claims 1-16.

30. An immunochemical reagent comprising a polypeptide encoded by the nucleotide sequence of any one of claims 1-16.
